(19) United States
(12) Patent Application Publication
(10) Pub. No.: US 2025/0034272 A1
(43) Pub. Date: Jan. 30, 2025

(54) COMPOSITIONS AND METHODS FOR TREATING SEVERE ASTHMA

(71) Applicant: Equillium, Inc., La Jolla, CA (US)

(72) Inventors: Stephen CONNELLY, La Jolla, CA (US); Cherie NG, San Diego, CA (US)

(21) Appl. No.: 18/739,089

(22) Filed: Jun. 10, 2024

Related U.S. Application Data
(63) Continuation of application No. 16/976,298, filed on Aug. 27, 2020, now abandoned.
(60) Provisional application No. 62/636,092, filed on Feb. 27, 2018.

(51) Int. Cl. C07K 16/28; A61K 31/137; A61K 31/46; A61K 31/573; A61P 11/06

(52) U.S. Cl. CPC C07K 16/2896; A61K 31/137; A61K 31/46; A61K 31/573; A61P 11/06

(57) ABSTRACT

The present disclosure provides compositions and methods related to treating, ameliorating, and preventing asthma, and in particular steroid resistant or refractory severe asthma, with an anti-CD6 antibody, or an antigen binding portion thereof, alone or in combination with other agents useful in treating asthma.

### CROSS-REFERENCE

This application is a continuation of U.S. application Ser. No. 16/976,298, filed Aug. 27, 2020, which is a § 371 U.S. national phase application of PCT/US2019/019872, filed Feb. 27, 2019, which claims priority to U.S. Provisional Application No. 62/636,092, filed Feb. 27, 2018, each of which application is incorporated by reference herein in its entirety.

### STATEMENT REGARDING SEQUENCE LISTING

The Sequence Listing XML associated with this application is provided in XML file format and is hereby incorporated by reference into the specification. The name of the XML file containing the Sequence Listing XML is EQIL_006_02US_ST26.xml. The XML file is 14,444 bytes, was created on Jun. 10, 2024, and is being submitted electronically via USPTO Patent Center.

### BACKGROUND

### Technical Field

The present invention relates to compositions and methods for treating severe asthma. Asthma is a disease in which (i) bronchoconstriction, (ii) excessive mucus production, and (iii) inflammation and swelling of airways occur, causing widespread but variable airflow obstruction thereby making it difficult for the asthma sufferer to breathe. Asthma is a chronic disorder, primarily characterized by persistent airway inflammation. However, asthma is further characterized by acute episodes of additional airway narrowing via contraction of hyper-responsive airway smooth muscle.

In asthma, chronic inflammatory processes in the airway play a central role in increasing the resistance to airflow within the lungs.

The chronic nature of asthma can also lead to remodeling of the airway wall (i.e., structural changes such as thickening or edema) which can further affect the function of the airway wall and influence airway hyper-responsiveness. Other physiologic changes associated with asthma include excess mucus production, and if the asthma is severe, mucus plugging, as well as ongoing epithelial denudation and repair. Epithelial denudation exposes the underlying tissue to substances that would not normally come in contact with them, further reinforcing the cycle of cellular damage and inflammatory response.

In susceptible individuals, asthma symptoms include recurrent episodes of shortness of breath (dyspnea), wheezing, chest tightness, and cough. Currently, asthma is managed by a combination of stimulus avoidance and pharmacology, depending primarily on the subtype of asthma that the patient has.

Asthma is heterogeneous disease classified by the National Asthma Education and Prevention Program (NAEPP) into a four main clinical categories based upon the severity of the asthma including: (1) intermittent asthma, (2) mild persistent asthma, (3) moderate persistent asthma, (4) and severe persistent asthma. The 2016 Global Initiative for Asthma (GINA), on the other hand, categorizes asthma severity as mild, moderate, or severe, with the severity being assessed based on the level of treatment required to control symptoms and exacerbations.

For example, NAEPP has the following clinical characterizations:

Intermittent asthma is characterized by:
Symptoms of cough, wheezing, chest tightness, or difficulty breathing less than twice a week; flare-ups that are brief, but intensity may vary; nighttime symptoms less than twice a month; no symptoms between flare-ups; lung function test FEV 1 is 80% or more above normal values; peak flow that has less than 20% variability am-to-am or am-to-pm, day-to-day (worldwide web page://emedicine.medscape.com/article/296301-guidelines#g2).

Mild persistent asthma is characterized by:
Symptoms of cough, wheezing, chest tightness, or difficulty breathing 3-6 times a week; flare-ups that may affect activity level; nighttime symptoms 3-4 times a month; lung function test FEV 1 is 80% or more above normal values; peak flow that has less than 20-30% variability (Id.).

Moderate persistent asthma is characterized by:
Symptoms of cough, wheezing, chest tightness, or difficulty breathing daily; flare-ups that may affect activity level; nighttime symptoms 5 or more times a month; lung function test FEV 1 is above 60% but below 80% of normal values; peak flow that has more than 30% variability (Id.).

Severe persistent asthma is characterized by:
Symptoms of cough, wheezing, chest tightness, or difficulty breathing that are continual; frequent nighttime symptoms; lung function test FEV 1 is 60% or less of normal values; peak flow has more than 30% variability (Id.).

Furthermore there are four distinct phenotypes of airway inflammation based on the types of inflammatory cells, or lack of them, in asthmatic airways: Eosinophilic inflammation, Neutrophillic inflammation, Mixed inflammation, and Paucigranulocytic inflammation. As is discussed further below, neutrophillic inflammation, mixed inflammation, and paucigranulocytic inflammation are forms of asthma that are non-eosinophilic.

Eosinophilic inflammation, often referred to as eosinophilic or allergic asthma, which is considered the product of a Th2-mediated inflammatory response (often referred to as Type 2 High) with the production of IL-4, IL-5, and IL-13 resulting in increased lung eosinophils. Eosinophils secrete additional cytokines that enhance the underlying inflammation and worsen the symptoms of asthma. Clinically, differentiation of eosinophilic asthma, as opposed to non-eosinophilic forms, is based primarily on the level of circulating eosinophils found in a blood sample, which is used as a surrogate to understanding the levels of eosinophils that are likely to be in the lung. Generally, patients are considered to have “low” blood eosinophil counts if they blood eosinophil counts ≤300 cells/μl. Such patients have non-eosinophilic asthma. Conversely, patients with blood eosinophil counts >300 cell/μl are considered eosinophilic.

Neutrophillic inflammation, often referred to as neutrophilic or non-allergic asthma, is considered the product of a Th1/Th17-mediated inflammatory response (often referred to as Type 2 low) with the production of IFN-γ, IL-6, IL-17 and IL-8 resulting in increased lung neutrophils. Neutrophils secrete additional cytokines that enhance the underlying inflammation and worsen the symptoms of asthma. Generally, patients with neutrophilic asthma manifest blood neutrophil counts >4600 cells/μl, for example (Vedel-Krogh, S. et al., Clin Chem. 2017 April;63 (4): 823-832; Schleich, F. et al., BMC Pulm Med. 2013; 13:11).

Mixed inflammation is a phenotype where patients can exhibit both eosinophilic and/or neutrophilic inflammation although the levels of either of these granulocytes may be low.

Paucigranulocytic inflammation is a phenotype where patients exhibit lung inflammation despite having normal levels of eosinophils and neutrophils. Also a form of non-allergic asthma, it is believed to be the product of a Th1/Th17-mediated inflammatory response (often referred to as Type 2 low) with the production of IFN-γ, IL-6 and IL-17. Notably, paucigranulocytic asthma patients typically have no detectable blood eosinophil counts.

The heterogeneity of asthma is illustrated in FIG. 7, which shows the relationship between inflammatory type (e.g., high or low Th2) and the current understanding of the various asthma phenotypes. Although the phenotypes overlap, Th2-mediated responses are shown on the left of the figure, and these correlated with allergic eosinophilic responses that are responsive to corticosteroid treatment. In contrast, non-allergic, non-eosinophilic responses on the right of the figure exhibit low Th2 responses, have high Th1 and or Th1/Th17 responses, are neutrophilic or paucigranulocytic, and do not exhibit responsiveness to corticosteroid treatment.

Current treatment/management options for asthma range from stimulus avoidance to pharmacological or even surgical interventions.

Stimulus avoidance is accomplished via systematic identification and minimization of contact with each type of stimuli. It may, however, be impractical and not always helpful to avoid all potential stimuli.

Asthma is managed pharmacologically by: (1) long term control through use of anti-inflammatories and long-acting bronchodilators and (2) short term management of acute exacerbations through use of short-acting bronchodilators (e.g., beta agonists). Both of these approaches require repeated and regular use of the prescribed drugs.

In a clinical setting, patients with asthma are most frequently tested for allergic or other forms of asthma by analysis of their blood eosinophil counts in order to determine the best course of action with respect to disease management. As is discussed further herein, steroid treatment is contraindicated for patients with low or no eosinophil counts in their blood. Although there is a spectrum in the art with respect to where exactly the cutoff for differentiating low vs normal or high blood eosinophil counts, as used herein, a “low” blood eosinophil count means that the subject has blood eosinophil counts ≤300 cells/μl. Such patients with low (or no) detectable blood eosinophils represent a high risk population of severe asthma patients that are in desperate need of an effective treatment agent.

Current treatments for asthma include long-acting beta agonists (LABAS), short-acting beta agonists (SABAs), such as albuterol (ProAir HFA, Proventil HFA, Ventolin HFA), Metaproterenol, Levalbuterol (Xopenex HFA), and Pirbuterol (Maxair), which are administered via quick-acting (rescue) inhaler or via nebulizers, which turns the medication into a mist that can be inhaled deep into the patient's lungs. Corticosteroids are also used to treat asthma. These medications, which are often inhaled or taken in pill form, help reduce lung inflammation and control asthma symptoms. Corticosteroids can also be given intravenously, typically to patients who are vomiting or under respiratory failure. Ipratropium (atrovent) is also sometimes used as a bronchodilator to treat a severe asthma attacks, especially if albuterol is not fully effective. Table 1 provides a list of various pharmacological agents that are either currently FDA approved for treating severe asthma or that are currently, or have previously been, under development for treating asthma.

TABLE 1
Agents Approved or Developed for Treating Severe Asthma

| Program | Company | Dev Stage | Target asthma population | Technology, MOA | Dosing |
|---|---|---|---|---|---|
| Xolair | Roche | Approved | Moderate/ | mAb, anti-IgE | SC, Q2W- |
| (omalizumab) | | | severe, | | Q4W |
| | | | persistent | | |
| Cinqair | Teva | Approved | Severe, | mAb, anti-IL-5 | IV, Q4W |
| (reslizumab) | | | eosinophilic | | |
| Nucala | GSK | Approved | Severe, | mAb, anti-IL-5 | SC, Q4W |
| (mepolizumab) | | | eosinophilic | | |
| Fasenra | AstraZeneca | Approved | Severe, | mAb, anti-IL-5 | SC, Q4W- |
| (benralizumab) | | | eosinophilic | | Q8W |
| Dupixent | Regeneron, | Phase 3 | Severe | mAb, anti-IL- | SC, Q2W |
| (dupilumab) | Sanofi | | | 4/IL-13 | |
| Fevipiprant | Novartis | Phase 3 | Moderate/ | Small | Oral, daily |
| | | | severe | molecule, | |
| | | | | CRTH2 | |
| | | | | antagonist | |
| Tezepelumab | Amgen | Phase 2/3 | Severe, | mAb, anti- | SC, Q4W |
| | | | uncontrolled | TSLP | |
| CJM112 | Novartis | Phase 2 | Moderate/ | mAb, anti- | SC |
| | | | severe, | IL17A | |
| | | | uncontrolled | | |
| MSTT1041A | Roche | Phase 2 | Severe, | mAb, anti-IL-33 | SC, Q4W |
| | | | uncontrolled | | |
| SAR440340/ | Regeneron, | Phase 2 | Moderate/ | mAb, anti-IL-33 | SC, Q2W |
| REGN3500 | Sanofi | | severe | | |
| GSK3772847 | GSK | Phase 2 | Moderate/ | mAb, anti-IL-33 | IV, Q4W |
| | | | severe | | |
| ANB020 | AnaptysBio | Phase 2 | Severe, | mAb, anti-IL-33 | SC |
| | | | eosinophilic | | |
| OC000459 | Chiesi/ | Phase 2 | Moderate/ | Small | Oral, daily |
| | Atopix | | severe | molecule, CRTH2 | |
| | | | | antagonist | |
| AZD1419 | AstraZeneca, | Phase 2 | Moderate/ | Oligonucleotide, | Inhaled, |
| | Dynavax | | severe | TLR-9 | QW |
| | | | | agonist | |
| Risankizumab | AbbVie, | Phase 2 | Severe, | mAb, anti-IL-23 | SC |
| | Boehringer | | persistent | | |
| | Ingelheim | | | | |
| ACT-774312 | Idorsia | Phase 1 | Moderate/ | Small | Oral |
| | | | severe, | molecule, | |
| | | | uncontrolled | CRTH2 | |
| | | | | antagonist | |
| PRS-060 | Pieris, | Phase 1 | Moderate/ | Biologic, IL-4 | Inhaled |
| | AstraZeneca | | severe | antagonist | |
| UCB4144/ | UCB, | Phase 1 | Moderate/ | Biologic, IL-13 | Inhaled |
| VR942 | Vectura | | severe | antagonist | |
| Lebrikizumab | Roche | Failed | Severe | mAb, anti-ll-13 | SC, Q4W |
| | | | Uncontrolled | | |
| Tralokinumab | AstraZeneca | Failed | Severe | mAb, anti-ll-13 | SC, Q2W |
| | | | Uncontrolled | | |

(As of January 2018)

Finally, if an asthma attack is life-threatening, intubation, mechanical ventilation, and oxygen may be needed to help the patient breathe while other medications are tried to bring the asthma under control.

However, currently available treatments all have deficiencies.

The difficulty involved in patient compliance with pharmacologic management and the difficulty of avoiding stimulus that triggers asthma are common barriers to successful asthma management. Moreover, many of these treatments focus on reducing symptoms of asthma, but do not get at the root causes of the disease. Thus, current management techniques are neither completely successful nor free from side effects.

High doses of corticosteroid anti-inflammatory drugs can have serious side effects that require careful management. In addition, some patients are resistant or refractory to steroid treatments. These patients make up an additional category of asthma patients, called severe asthma (SA) patients, which are of huge clinical concern because of the severity of their disease and the lack of their response to traditional asthma treatments. Indeed, although only 5% to 10% of asthmatics have steroid resistant or refractory disease, care for these patients accounts for as much as 50%-80% of all asthma related health care costs in the United States, Europe, and Australia due to their frequent hospitalizations and need for emergency care (Hansbro et al., Immunological Reviews. 2017;278:41-62.

Severe asthma is most-commonly associated with non-allergic Th1/Th17 phenotypes such as neutrophilic or paucigranulocytic asthma, or a mixed phenotype of Th1/Th17-neutrophilic/Th2-eosinophilic asthma, wherein Th17 cells and neutrophils respond poorly, and sometimes not at all, to corticosteroids (FIG. 7).

Whereas allergic, Th2-mediated, eosinophilic asthma is typically responsive to corticosteroid treatment (FIG. 7).

Accordingly, a need exists for new asthma treatments that are selective in treating the underlying pathology of the disease. A need also exists for new asthma treatments that are effective in treating the steroid resistant or refractory forms of asthma (i.e., SA). The present invention provides compositions and methods for treating, preventing, and attenuating severe asthma, and in particular embodiments severe asthma characterized by low or no blood eosinophil counts.

### SUMMARY OF THE EMBODIMENTS

The present disclosure relates to, inter alia, the treatment, prevention, or attenuation of asthma comprising administering an anti-CD6 antibody to a subject. In particular embodiments, the anti-CD6 antibody is EQ001. In some embodiments, the asthma is severe asthma. In some embodiments, the asthma is characterized by low or no eosinophils. In some embodiments, the asthma is a neutrophilic asthma. In some embodiments, the asthma is a paucigranulocytic asthma. In some embodiments, the asthma is a mixed inflammation asthma. In certain embodiments, the asthma is not allergic asthma. In certain embodiments, the asthma is not eosinophilic asthma.

In some embodiments, the present invention provides a method of inhibiting T cell-mediated pulmonary inflammation in a subject that has asthma comprising, administering to a subject an anti-CD6 antibody, or an antigen binding fragment thereof, wherein the anti-CD6 antibody, or the antigen binding fragment thereof, comprises heavy and light chain variable regions comprising amino acid sequences as set forth in SEQ ID NOs: 1 and 2.

In some embodiments, the present invention provides a method of inhibiting T cell-mediated pulmonary inflammation in a subject that has asthma comprising, administering to a subject an anti-CD6 antibody, or an antigen binding fragment thereof.

In some embodiments, the present invention provides a method of preventing or attenuating the migration of a T cell into and through a pulmonary tissue in response to an asthma-inducing antigen comprising, administering to a subject an anti-CD6 antibody, or an antigen binding fragment thereof.

In some embodiments, the present invention provides a method of modulating or attenuating a symptom or the severity of asthma comprising, administering to a subject an anti-CD6 antibody, or an antigen binding fragment thereof.

In some embodiments, the present invention provides a method of modulating or attenuating a symptom or the severity of asthma comprising, contacting a T-cell with an anti-CD6 antibody, or an antigen binding fragment thereof.

In some embodiments, the asthma is severe asthma. In some embodiments, the asthma is characterized by low or no blood eosinophils. In some embodiments, the asthma is refractory to steroid treatment. In some embodiments, the asthma is a neutrophilic asthma. In some embodiments, the asthma is a mixed inflammation asthma. In some embodiments, the asthma is paucigranulocytic.

In some embodiments, the anti-CD6 antibody, or the antigen binding fragment thereof, binds to a CD6 protein on the surface of a T cell. In some embodiments, the T cell is a Th1, Th17, or a Th1 and Th17 T cell.

In some embodiments, the anti-CD6 antibody, or the antigen binding fragment thereof, is EQ001, or an antigen binding fragment of EQ001. In some embodiments, the anti-CD6 antibody, or the antigen binding fragment thereof, binds to domain 1 or 3 on CD6. In some embodiments, the anti-CD6 antibody, or the antigen binding fragment thereof, binds to domain 3 on CD6. In some embodiments, the binding of the anti-CD6 antibody, or the antigen binding fragment thereof, to the CD6 protein on the surface of a T cell modulates the activity and/or migration of the T cell. In some embodiments, the anti-CD6 antibody, or the antigen binding fragment thereof, is a humanized antibody. In some embodiments, the anti-CD6 antibody, or the antigen binding fragment thereof, is selected from the group consisting of: UMCD6 mAb, Itolizumab (EQ001), an anti-CD6 antibody described on Table 2, and an anti-CD6 antibody disclosed herein. In some embodiments, the anti-CD6 monoclonal antibody is an antibody produced by secreting hybridoma IOR-TIA deposited with the ECACC as deposit No. ECACC 96112640; an antibody having the same sequence as said antibody produced by said secreting hybridoma; or an antibody having the same CDR sequences of said antibody produced by said secreting hybridoma.

In some particular embodiments, any one of the methods disclosed herein comprises administering EQ001. In some particular embodiments, any one of the methods disclosed herein comprises administering an antigen binding fragment EQ001. In some embodiments, the antigen binding fragment is selected from an Fv, Fab, CDR1, CDR2, CDR3, combination of CDRs, variable region, heavy chain(s), and light chain(s).

In some embodiments, the anti-CD6 antibody, or the antigen binding fragment thereof, comprises one or more CDR sequence selected from SEQ ID NOS: 5-10. In some embodiments, the anti-CD6 antibody, or the antigen binding fragment thereof, comprises heavy and light chain variable regions comprising amino acid sequences as set forth in SEQ ID NOs: 1 and 2. In some embodiments, SEQ ID NOs: 1 and 2 are encoded by SEQ ID NOs: 3 and 4 respectively. In some embodiments, the anti-CD6 antibody, or the antigen binding fragment thereof, comprises a VH sequence that is at least 80% identical to the amino acid sequence as set forth in SEQ ID NO: 1. In some embodiments, the anti-CD6 antibody, or the antigen binding fragment thereof, comprises a VK sequence that is at least 80% identical to the amino acid sequence as set forth in SEQ ID NO: 2. In some embodiments, the anti-CD6 antibody, or the antigen binding fragment thereof, comprises a VH sequence that is at least 80% identical to the amino acid sequence as set forth in SEQ ID NO: 1 and a VK sequence that is at least 80% identical to the amino acid sequence as set forth in SEQ ID NO: 2.

In some embodiments, any one of the methods of the present disclosure further comprise administering one or more additional agent capable of treating, preventing, or attenuating one or more asthma related symptom. In some embodiments, the additional agent comprises an agent that is capable of modulating the immune system. In some embodiments, the additional agent comprises an agent that is immunosuppressant. In some embodiments, the additional agent comprises a long-acting beta agonist, a short-acting beta agonist, or a combination thereof. In some embodiments, the additional agent comprises albuterol. In some embodiments, the albuterol is administered in a dosage form selected from: an aerosol powder; a solution; a capsule; and a powder suspension. In some embodiments, the additional agent comprises a corticosteroid. In some embodiments, the corticosteroid is administered as an inhaled formulation. In some embodiments, the corticosteroid is administered in a dosage form selected from a tablet, a delayed release capsule; an extended release tablet; an extended release capsule; a syrup; a solution; an elixir; a suspension; a delayed release tablet; a liquid; and a disintegrating tablet. In some embodiments, the additional agent comprises Ipratropium. In some embodiments, the Ipratropium is administered in a spray dosage form.

In some embodiments, any one of the methods of the present disclosure further comprises administration of intubation, mechanical ventilation, and/or oxygen therapy.

In some embodiments, in any one of the methods of the present disclosure the anti-

CD6 antibody, or antigen binding fragment thereof, is administered as a pharmaceutical composition comprising one or more pharmaceutically acceptable salts, excipients or vehicles. In some embodiments, the composition comprises one or more agent selected from the group consisting of carriers, excipients, diluents, antioxidants, preservatives, coloring, flavoring and diluting agents, emulsifying agents, suspending agents, solvents, fillers, bulking agents, buffers, delivery vehicles, tonicity agents, cosolvents, wetting agents, complexing agents, buffering agents, antimicrobials, and/or surfactants.

In some embodiments, the present invention provides a method of inhibiting T cell-mediated pulmonary inflammation in a subject that has asthma comprising, administering to a subject an anti-CD6 antibody, or an antigen binding fragment thereof, wherein the anti-CD6 antibody, or the antigen binding fragment thereof, comprises heavy and light chain variable regions comprising amino acid sequences as set forth in SEQ ID NOs: 1 and 2, and wherein the asthma is characterized by low or no blood eosinophils. In some embodiments, the asthma is resistant or refractory to steroid treatment. In some embodiments, the asthma is a neutrophilic asthma. In some embodiments, the asthma is a mixed inflammation asthma. In some embodiments, the asthma is paucigranulocytic. In some embodiments, the T cell is selected from (i) a Th1 T cell, (ii) a Th17 T cell, or (iii) a Th1 and Th17 T cell. In some embodiments, the subject has blood eosinophils counts ≤300 cells/μl. In some embodiments, the subject has a non-allergic asthma. In some embodiments, the anti-CD6 antibody is EQ001.

In some embodiments, the present invention provides a method of inhibiting T cell-mediated pulmonary inflammation in a subject that has asthma comprising, administering to a subject an anti-CD6 antibody, or an antigen binding fragment thereof, wherein the asthma is characterized by low or no blood eosinophils.

In some embodiments, the present invention provides a method of preventing or attenuating the migration of a T cell into and through a pulmonary tissue in response to an asthma-inducing antigen, wherein the asthma is characterized by low or no blood eosinophils, comprising administering to a subject an anti-CD6 antibody, or an antigen binding fragment thereof.

In some embodiments, the present invention provides a method of modulating or attenuating a symptom or the severity of asthma comprising, administering to a subject an anti-CD6 antibody, or an antigen binding fragment thereof when the asthma is characterized by low or no blood cosinophils.

In some embodiments, the present invention provides a method of modulating or attenuating a symptom or the severity of asthma, comprising contacting a T-cell with an anti-CD6 antibody, or an antigen binding fragment thereof, wherein the asthma is characterized by low or no blood cosinophils.

In some embodiments, the asthma is resistant or refractory to steroid treatment. In some embodiments, the asthma is a neutrophilic asthma. In some embodiments, the asthma is a mixed inflammation asthma. In some embodiments, the asthma is paucigranulocytic. In some embodiments, the T cell is selected from (i) a Th1 T cell, (ii) a Th17 T cell, or (iii) a Th1 and Th 17 T cell. In some embodiments, the subject has blood eosinophils counts ≤300 cells/μl. In some embodiments, the subject has a non-allergic asthma. In some embodiments, the asthma is severe asthma. In some embodiments, the asthma is severe asthma. In some embodiments, the anti-CD6 antibody or an antigen binding fragment thereof is EQ001 or an antigen binding fragment thereof. In some embodiments, the anti-CD6 antibody is EQ001. In some embodiments, the anti-CD6 antibody, or the antigen binding fragment thereof, binds to domain 1 or 3 on CD6. In some embodiments, the anti-CD6 antibody, or the antigen binding fragment thereof, binds to domain 3 on CD6. In some embodiments, the anti-CD6 antibody, or the antigen binding fragment thereof, is selected from the group consisting of: UMCD6 mAb, Itolizumab (EQ001), an anti-CD6 antibody described on Table 2, and an anti-CD6 antibody disclosed herein. In some embodiments, the anti-CD6 monoclonal antibody is an antibody produced by secreting hybridoma IOR-TIA deposited with the ECACC as deposit No. ECACC 96112640; an antibody having the same sequence as said antibody produced by said secreting hybridoma; or an antibody having the same CDR sequences of said antibody produced by said secreting hybridoma. In some embodiments, the anti-CD6 antibody, or the antigen binding fragment thereof, comprises one or more CDR sequence selected from SEQ ID NOS: 5-10. In some embodiments, the anti-CD6 antibody, or the antigen binding fragment thereof, comprises heavy and light chain variable regions comprising amino acid sequences as set forth in SEQ ID NOs: 1 and 2. In some embodiments, SEQ ID NOs: 1 and 2 are encoded by SEQ ID NOs: 3 and 4 respectively. In some embodiments, the anti-CD6 antibody, or the antigen binding fragment thereof, comprises a VH sequence that is at least 80% identical to the amino acid sequence as set forth in SEQ ID NO: 1. In some embodiments, the anti-CD6 antibody, or the antigen binding fragment thereof, comprises a VK sequence that is at least 80% identical to the amino acid sequence as set forth in SEQ ID NO: 2. In some embodiments, the anti-CD6 antibody, or the antigen binding fragment thereof, comprises a VH sequence that is at least 80% identical to the amino acid sequence as set forth in SEQ ID NO: 1 and a VK sequence that is at least 80% identical to the amino acid sequence as set forth in SEQ ID NO: 2. In some embodiments, the antigen binding fragment is selected from an Fv, Fab, CDR1, CDR2, CDR3, combination of CDRs, variable region, heavy chain(s), and light chain(s). In some embodiments, the anti-CD6 antibody, or the antigen binding fragment thereof, binds to a CD6 protein on the surface of a T cell. In some embodiments, the binding of the anti-CD6 antibody, or the antigen binding fragment thereof, to the CD6 protein on the surface of a T cell modulates the activity and/or migration of the T cell.

In some embodiments, such methods further comprises administering one or more additional agent capable of treating, preventing, or attenuating one or more asthma related symptom. In some embodiments, the additional agent comprises an agent that is capable of modulating the immune system. In some embodiments, the additional agent comprises an agent that is immunosuppressant. In some embodiments, the additional agent comprises a long-acting beta agonist, a short-acting beta agonist, or a combination thereof. In some embodiments, the additional agent comprises albuterol. In some embodiments, the albuterol is administered in a dosage form selected from: an aerosol powder; a solution; a capsule; and a powder suspension. In some embodiments, the additional agent comprises a corticosteroid. In some embodiments, the corticosteroid is administered as an inhaled formulation. In some embodiments, the additional agent comprises Ipratropium. In some embodiments, the Ipratropium is administered in a spray dosage form. In some embodiments, the method further comprises administration of intubation, mechanical ventilation, and/or oxygen therapy. In some embodiments, the anti-CD6 antibody, or antigen binding fragment thereof, is administered as a pharmaceutical composition comprising one or more pharmaceutically acceptable salts, excipients or vehicles. In some embodiments, the composition comprises one or more agent selected from the group consisting of carriers, excipients, diluents, antioxidants, preservatives, coloring, flavoring and diluting agents, emulsifying agents, suspending agents, solvents, fillers, bulking agents, buffers, delivery vehicles, tonicity agents, cosolvents, wetting agents, complexing agents, buffering agents, antimicrobials, and/or surfactants.

### BRIEF DESCRIPTION OF THE SEQUENCES

SEQ ID NO: 1: Amino acid sequence of EQ001 VH sequence.

SEQ ID NO: 2: Amino acid sequence of EQ001 VK sequence.

SEQ ID NO: 3: Nucleotide (DNA) sequence of EQ001 VH sequence.

SEQ ID NO: 4: Nucleotide (DNA) sequence of EQ001 VK sequence.

SEQ ID NO: 5: Amino acid sequence of EQ001 VH CDR1

SEQ ID NO: 6: Amino acid sequence of EQ001 VH CDR2

SEQ ID NO: 7: Amino acid sequence of EQ001 VH CDR3

SEQ ID NO: 8: Amino acid sequence of EQ001 VK CDR1

SEQ ID NO: 9: Amino acid sequence of EQ001 VK CDR2

SEQ ID NO: 10: Amino acid sequence of EQ001 VK CDR3

### BRIEF DESCRIPTION OF THE DRAWINGS

FIG. 1. Sequences of the EQ001 anti-CD6 antibody. FIG. 1A: Nucleotide sequences of the variable heavy (VH) and light (VK) chains of EQ001 derived from plasmid and genomic DNA. FIG. 1B: Amino acid sequences of the VH and VK of EQ001.

FIG. 2. CD6⁺ cells are present at high levels in the lung of a severe asthma patient. Left column: ALCAM is expressed in the lamina propria of fatal asthma lungs (bottom left), but not in the lamina propria of normal lungs (top left). Center column: CD6⁺ cells are increased in fatal asthma lungs (bottom center) as compared to normal lungs (top center). Right column: CD6⁺ cells colocalize with ALCAM expressing lamina propria in asthma lungs (bottom right).

FIG. 3. De novo bioinformatics-based analysis comparing expression of CD4, CD6, and various Th17 markers in control vs. moderate vs. severe asthma patients using a publicly available RNASeq dataset generated from cells collected by bronchiolar lavage. FIG. 3A: Significant differences in levels of CD4 expression, with severe asthma patients expressing significantly higher levels of CD4. FIG. 3B: Significant differences in levels of CD6 expression, with severe asthma patients expressing significantly higher levels of CD6. FIGS. 3C to 3G. Significant differences in levels of Th17 marker expression, with severe asthma patients expressing significantly higher levels of CD6. FIG. 3C: CCR6 expression; FIG. 3D: CCR4 expression; FIG. 3E: KLRB1 expression; FIG. 3F: IL-17A expression; FIG. 3G: IL-17F expression.

FIG. 4. CD6 is Highest in a Cluster of Severe Asthma Patients. FIG. 4A: Unsupervised cluster analysis based on expression of >1000 genes (regardless of asthma severity) show asthma patients group into 4 main clusters. FIG. 4B: Each cluster exhibited a different average expression of CD6 with the highest expression occurring in cluster 3 which is composed of only severe asthma patients, suggesting there is a subset of severe asthma patients with high target.

FIG. 5. CD6 blockade in murine model of allergic asthma decreases Th2 cytokines levels in bronchiolar lavage fluid. FIG. 5A: Table showing treatment groups and dose regiment. FIG. 5B: Illustration of dose regiment. FIG. 5C. CD6 blockade with mCD6D1 anti-CD6 antibody during challenge resulted in decreased levels of Th2 cytokines, IL-4, IL-5, and IL-13 in bronchiolar lavage fluid (BALF) and a modest reduction of these cytokines in lung cells (data not shown).

FIG. 6. CD6 blockade in classic Th2 model of OVA vaccination inhibits Th2-driven IgE production. FIG. 6A: Table showing treatment groups and dose regiment. FIG. 6B: Illustration of dose regiment. FIG. 6C. Prophylactic CD6 blockade with mCD6D1 anti-CD6 antibody inhibited OVA-specific IgE production, demonstrating the effect of the CD6 pathway on Th2 responses.

FIG. 7. Example shows asthma phenotypes as they relate to inflammatory type (Th2 high or low) and other variables. CS=corticosteroids; GM-CSF=granulocyte-macrophage colony-stimulating factor.

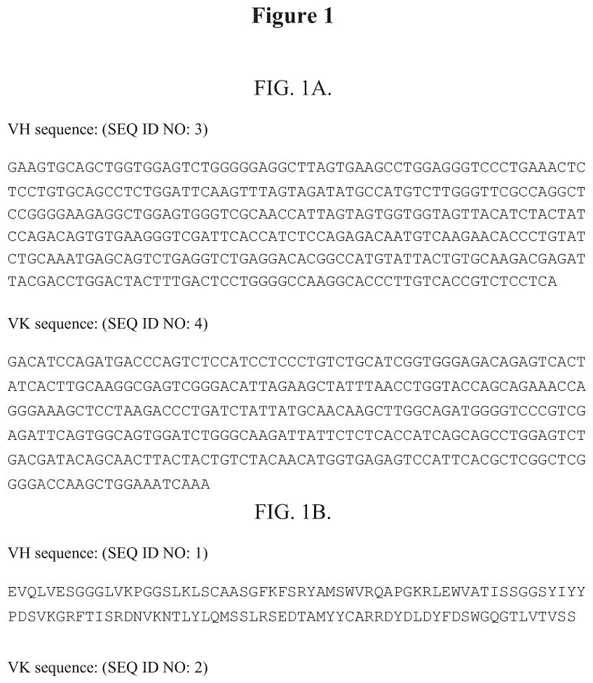

### DETAILED DESCRIPTION

The practice of the present invention will employ, unless indicated specifically to the contrary, conventional methods of molecular biology and recombinant DNA techniques within the skill of the art, many of which are described below for the purpose of illustration. Such techniques are explained fully in the literature. See, e.g., Sambrook, et al., Molecular Cloning: A Laboratory Manual (3^{rd} Edition, 2000); DNA Cloning: A Practical Approach, vol. I & II (D. Glover, ed.); Oligonucleotide Synthesis (N. Gait, ed., 1984); Oligonucleotide Synthesis: Methods and Applications (P. Herdewijn, ed., 2004); Nucleic Acid Hybridization (B. Hames & S. Higgins, eds., 1985); Nucleic Acid Hybridization: Modern Applications (Buzdin and Lukyanov, eds., 2009); Transcription and Translation (B. Hames & S. Higgins, eds., 1984); Animal Cell Culture (R. Freshney, ed., 1986); Freshney, R. I. (2005) Culture of Animal Cells, a Manual of Basic Technique, 5^{th} Ed. Hoboken NJ, John Wiley & Sons; B. Perbal, A Practical Guide to Molecular Cloning (3^{rd} Edition 2010); Farrell, R., RNA Methodologies: A Laboratory Guide for Isolation and Characterization (3rd Edition 2005). Poly(ethylene glycol), Chemistry and Biological Applications, ACS, Washington, 1997; Veronese, F., and J. M. Harris, Eds., Peptide and protein PEGylation, Advanced Drug Delivery Reviews, 54(4) 453-609 (2002); Zalipsky, S., et al., “Use of functionalized Poly(Ethylene Glycols) for modification of polypeptides” in Polyethylene Glycol Chemistry: Biotechnical and Biomedical Applications. The publications discussed above are provided solely for their disclosure before the filing date of the present application. Nothing herein is to be construed as an admission that the invention is not entitled to antedate such disclosure by virtue of prior invention.

### Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e., to at least one) of the grammatical object of the article. By way of example, “an element” means one element or more than one element.

The term “and/or” is used in this disclosure to mean either “and” or “or” unless indicated otherwise.

The term “e.g.” is used herein to mean “for example,” and will be understood to imply the inclusion of a stated step or element or group of steps or elements but not the exclusion of any other step or element or group of steps or elements.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

The term “administering”, as used herein, refers to any mode of transferring, delivering, introducing, or transporting matter such as a compound, e.g. a pharmaceutical compound, or other agent such as an antigen, to a subject. Modes of administration include oral administration, topical contact, intravenous, intraperitoneal, intramuscular, intranasal, or subcutaneous administration. Administration “in combination with” further matter such as one or more therapeutic agents includes simultaneous (concurrent) and consecutive administration in any order.

The term “binding partner” as used herein refers to matter, such as a molecule, in particular a polymeric molecule, that can bind a nucleic acid molecule such as a DNA or an RNA molecule, including an mRNA molecule, as well as a peptide, a protein, a saccharide, a polysaccharide or a lipid through an interaction that is sufficient to permit the agent to form a complex with the nucleic acid molecule, peptide, protein or saccharide, a polysaccharide or a lipid, generally via non-covalent bonding. In some embodiments the binding partner is an immunoglobulin or a proteinaceous binding molecule with immunoglobulin-like functions as defined below. In some embodiments the binding partner is an aptamer. In some embodiments a binding partner is specific for a particular target. In some embodiments a binding partner includes a plurality of binding sites, each binding site being specific for a particular target. As an illustrative example, a binding partner may be a proteinaceous agent with immunoglobulin-like functions with two binding sites. It may for instance be antigen binding fragment of an antibody. It may for instance be a bispecific diabody, such as a bispecific single chain diabody.

The term “carrier”, as used in this disclosure, encompasses carriers, excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body of a subject.

As used herein, the term “chimeric antibody” refers to an immunoglobulin polypeptide or domain antibody that includes sequences from more than one species. In a chimeric antibody a heavy chain or a light chain may contain a variable region sequence from one species such as human and a constant region sequence from another species such as mouse. As an example, a “chimeric antibody” may be an immunoglobulin that has variable regions derived from an animal antibody, such as a rat or mouse antibody, fused to another molecule, for example, the constant domains derived from a human antibody. The term “chimeric antibody” is intended to encompass antibodies in which: (i) the heavy chain is chimeric but the light chain comprises V and C regions from only one species; (ii) the light chain is chimeric but the heavy chain comprises V and C regions from only one species; and (iii) both the heavy chain and the light chain are chimeric.

An “effective amount,” when used in connection with a compound, is an amount of the compound, such as an anti-CD6 antibody (e.g., EQ001), needed to elicit a desired response. In some embodiments, the desired response is a biological response, e.g., in a subject. In some embodiments, the compound (e.g., an anti-CD6 antibody) may be administered to a subject in an effective amount to effect a biological response in the subject. In some embodiments, the effective amount is a “therapeutically effective amount.”

The terms “therapeutically effective amount” and “therapeutic dose” are used interchangeably herein to refer to an amount of a compound, such as an anti-CD6 antibody (e.g., EQ001), which is effective following administration to a subject for treating a disease or disorder in the subject as described herein.

The term “prophylactically effective amount” is used herein to refer to an amount of a compound, such as an anti-CD6 antibody (e.g., EQ001), which is effective following administration to a subject, for preventing or delaying the onset of a disease or disorder in the subject as described herein.

In this regard, a “humanized antibody” as used herein is an immunoglobulin polypeptide or domain antibody containing structural elements of a human antibody and the antigen binding site of a non-human antibody. “Humanized antibodies” contain a minimal number of residues from the non-human antibody from which they are derived. For instance, they may contain only the CDR regions of the non-human antibody, or only those residues that make up the hypervariable regions of the non-human antibody. They may also contain certain residues from outside the variable regions of the non-human polypeptide, such as residues that are necessary to mimic the structure of the non-human antibody or to minimize steric interference. Typically a humanized antibody contains a human framework, at least one CDR from a non-human antibody, with any constant region present being substantially identical to a human immunoglobulin constant region, i.e., at least about 85-90%, such as at least 95% identical. Hence, in some instances all parts of a humanized immunoglobulin, except possibly the CDRs, are substantially identical to corresponding parts of one or more native human immunoglobulin sequences. In addition, humanized antibodies may contain residues that do not correspond to either the human or the non-human antibodies.

As used herein, the term “antibody fragment” refers to any form of an antibody other than the full-length form. Antibody fragments herein include antibodies that are smaller components that exist within full-length antibodies, and antibodies that have been engineered. Antibody fragments include, but are not limited to, Fv, Fc, Fab, and (Fab′)2, single chain Fv (scFv), diabodies, triabodies, tetrabodies, bifunctional hybrid antibodies, CDR1, CDR2, CDR3, combinations of CDRs, variable regions, framework regions, constant regions, heavy chains, light chains, alternative scaffold non-antibody molecules, and bispecific antibodies. Unless specifically noted otherwise, statements and claims that use the term “antibody” or “antibodies” may specifically include “antibody fragment” and “antibody fragments.”

The term “asthma” has its ordinary scientific meaning and includes intermittent asthma, mild persistent asthma, moderate persistent asthma, and severe persistent asthma.

The term “severe asthma” is used herein to describe a separate category of asthma in which disease symptoms are poorly controlled by steroids. Severe asthma includes asthma that is steroid resistant and/or refractory to corticosteroids (CS). In some embodiments, severe asthma (SA) is driven primarily by a neutrophilic Th1/Th17 T cell-mediated response. In some embodiments, SA is driven primarily by a paucigranulocytic Th1/Th17 T cell-mediated response. In some embodiments, SA is also resistant to one or more other asthma therapeutic. For example, in some embodiments, SA is also resistant to one or more of a SABA and/or a LABA. In some embodiments, severe asthma may be characterized by having a neutrophilic Th1/Th17 T cell mediated response, a paucigranulocytic Th1/Th17 T cell-mediated response, or a combined neutrophilic Th1/Th17 and eosinophilic Th2 T cell mediated response. In certain embodiments, severe asthma may comprise a neutrophilic T cell response, but not an eosinophilic T cell response. In certain embodiments, severe asthma may comprise a paucigranulocytic T cell response, but not an eosinophilic T cell response.

The term “steroid resistant asthma” is used herein to describe an asthma for which steroid treatment (e.g., a corticosteroid) has limited efficacy. Thus, treatment of a steroid resistant asthma with a steroid (e.g., a corticosteroid) would produce very little detectable therapeutic benefit. In some cases, such a treatment produces substantially no therapeutic benefit.

The term “steroid refractory asthma” is used herein to describe an asthma for which steroid treatment (e.g., a corticosteroid) has no efficacy. Thus, treatment of a steroid refractory asthma with a steroid (e.g., a corticosteroid) would produce no detectable therapeutic benefit.

The term “LABA” means long-acting beta agonist. LABAs are known in the art and include, without limitation, formoterol fumarate; salmeterol xinafoate

The term “SABA” means short-acting beta agonist. SABAs are known in the art and include, without limitation, albuterol (e.g., albuterol sulfate, albuterol sulfate HFA, albuterol sulfate inhalation solution, albuterol sulfate nebulizer solution, and levalbuterol hydrochloride), metaproterenol, pirbuterol (e.g., pirbuterol acetate); isoetharine hydrochloride; isoproterenol hydrochloride; and terbutaline sulfate.

The term “VH” is used herein to denote the variable heavy chain of an antibody. The term “VK” is used herein to denote the variable light chain of an antibody.

The term “Antigen binding fragment” in reference to an antibody refers to any antibody fragment that retains binding affinity for an antigen to which the parent full length antibody binds, and antigen binding fragments include, but are not limited to, Fv, Fab, (Fab′)2, scFv, diabodies, triabodies, tetrabodies, bifunctional hybrid antibodies, CDR1, CDR2, CDR3, combinations of CDRs, variable regions, heavy chains, light chains, and bispecific antibodies.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The term “modulating” includes “increasing,” “enhancing” or “stimulating,” as well as “decreasing” or “reducing,” typically in a statistically significant or a physiologically significant amount as compared to a control. An “increased,” “stimulated” or “enhanced” amount is typically a “statistically significant” amount, and may include an increase that is 1.1, 1.2, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the amount produced by no composition (e.g., in the absence of any of the anti-CD6 antibodies of the invention) or a control composition, sample or test subject. A “decreased” or “reduced” amount is typically a “statistically significant” amount, and may include a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% decrease in the amount produced by no composition (the absence of an agent or compound) or a control composition, including all integers in between.

The terms “polypeptide” and “protein” are used interchangeably herein to refer to a polymer of amino acid residues and to variants and synthetic analogues of the same. Thus, these terms apply to amino acid polymers in which one or more amino acid residues are synthetic non-naturally-occurring amino acids, such as a chemical analogue of a corresponding naturally-occurring amino acid, as well as to naturally-occurring amino acid polymers.

A “subject,” or “patient” as used herein, includes any animal that exhibits a symptom, or is at risk for exhibiting a symptom, which can be treated or diagnosed with an anti-CD6 antibody, or an antigen binding fragment thereof. Suitable subjects (patients) includes, preferably, human patients. Suitable subjects also include laboratory animals (such as mouse, rat, rabbit, or guinea pig), farm animals (such as pig, horse, cow), and domestic animals or pets (such as a cat or dog). Non-human primates (such as a monkey, chimpanzee, baboon or rhesus) are also included.

“Substantially” or “essentially” means nearly totally or completely, for instance, 95% or greater of some given quantity.

“Treatment” or “treating,” as used herein, includes any desirable effect on the symptoms or pathology of a disease or condition, and may include even minimal changes or improvements in one or more measurable markers of the disease or condition being treated. “Treatment” or “treating” does not necessarily indicate complete eradication or cure of the disease or condition, or associated symptoms thereof. The subject receiving this treatment is any subject in need thereof. Exemplary markers of clinical improvement will be apparent to persons skilled in the art.

Unless defined otherwise, all technical and scientific terms used herein have the same meaning as commonly understood by one of ordinary skill in the art to which the invention belongs. Although any methods, compositions, reagents, cells, similar or equivalent to those described herein can be used in the practice or testing of the invention, the preferred methods and materials are described herein. All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

### Overview

The present disclosure relates to the treatment, prevention, or attenuation of severe asthma comprising administering an anti-CD6 antibody to a subject.

CD6 is an important cell surface protein predominantly expressed by human T cells and a subset of B cells, as well as by some B cell chronic lymphocytic leukemias and neurons [Aruffo et al., J. Exp. Med. 1991, 174:949; Kantoun et al., J. Immunol. 1981, 127:987; Mayer et al., J. Neuroimmunol. 1990. 29:193]. CD6 is a member of a large family of proteins characterized by having at least one domain homologous to the scavenger receptor cysteine-rich domain (SRCR) of type I macrophages [Matsumoto, et al., J. Exp. Med. 1991, 173:55 and Resnick et al., Trends Biochem. Sci. 1994, 19:5]. Other members of this family include CD5 [Jones et al., Nature. 1986, 323:346]; cyclophilin C [Friedman et al. 1993, PNAS 90:6815]; complement factor I, which binds activated complement proteins C3b and C4b [Goldberger, et al., J. Biol. Chem. 1987, 262:10065]; bovine WC-1 expressed by .tau./.delta. T cells [Wijingaard et al., J. Immunol. 1992, 149:3273] and M130 [Law et al., Eur J. Immunol. 1993, 23:2320], a macrophage activation marker.

The extracellular domain of the mature CD6 protein is composed of three SRCR domains (hereinafter designated D1, D2, and D3). D3 corresponding to the membrane proximal SRCR domain followed by a short 33-amino-acid stalk region. These extracellular domains are anchored to the cell membrane via a short transmembrane domain followed by a cytoplasmic domain of variable length [Aruffo et al., J. Exp. Med. 1991, 174:949].

Studies using CD6-immunoglobulin fusion proteins, containing selected extracellular domains of CD6 fused to human IgG1 constant domains (CD6-Rgs), led to the identification and cloning of a CD6 ligand, designated “activated leukocyte cell adhesion molecule” (ALCAM) also known as CD166 [Patel, et al., J. Exp. Med. 1995. 181:1563-1568; Bowen et al., J. Exp. Med 1995, 181:2213-2220].

ALCAM, is a 100-105 kD type I transmembrane glycoprotein that is a member of the immunoglobulin superfamily and comprises five extracellular immunoglobulin domains (2 NH2-terminal, membrane-distal variable-(V)-type (V1, V2 or D1, D2) and 3 membrane-proximal constant-(C2)-type Ig folds) [C1, C2, C3], a transmembrane region, and a short cytoplasmic tail. The N-terminal domain (D1) is exclusively involved in ligand binding, whereas membrane proximal domains (C2, C3 or D4, D5) are required for homophilic interactions.

ALCAM binds to domain 3 of CD6 corresponding to the membrane proximal SRCR domain [Whitney, et. al., J. Biol. Chem 1995, 270:18187-18190].

CD6 is expressed on the surface of T cells including CD4⁺ T cells and can play a role in T cell activation, differentiation, survival and migration. However, a role for CD6⁺ T cells in the pathogenesis of severe, non-allergic asthma has not previously been reported.

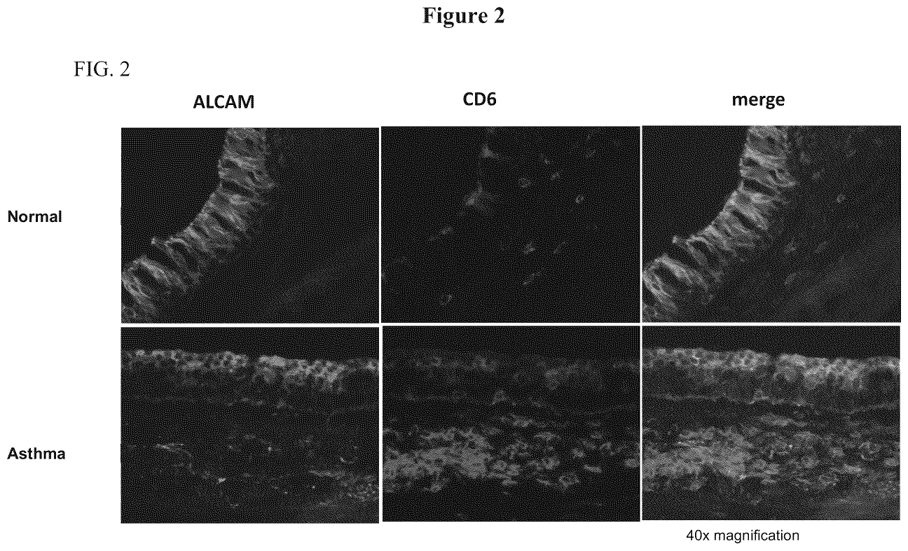

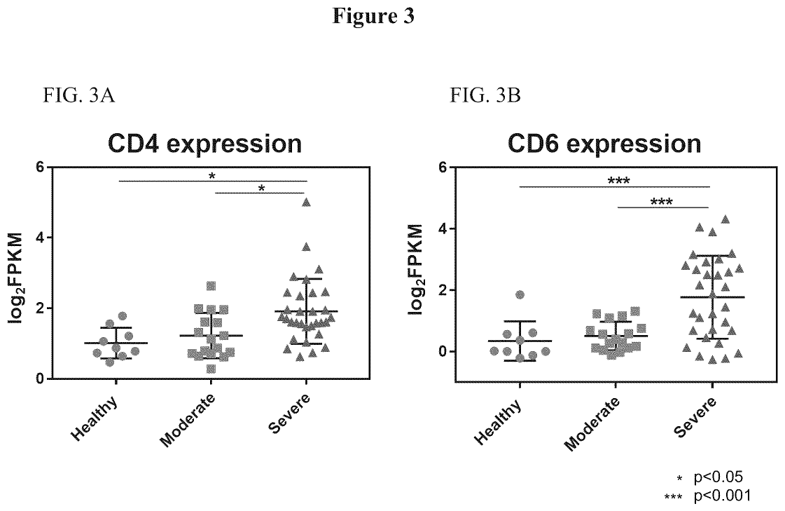

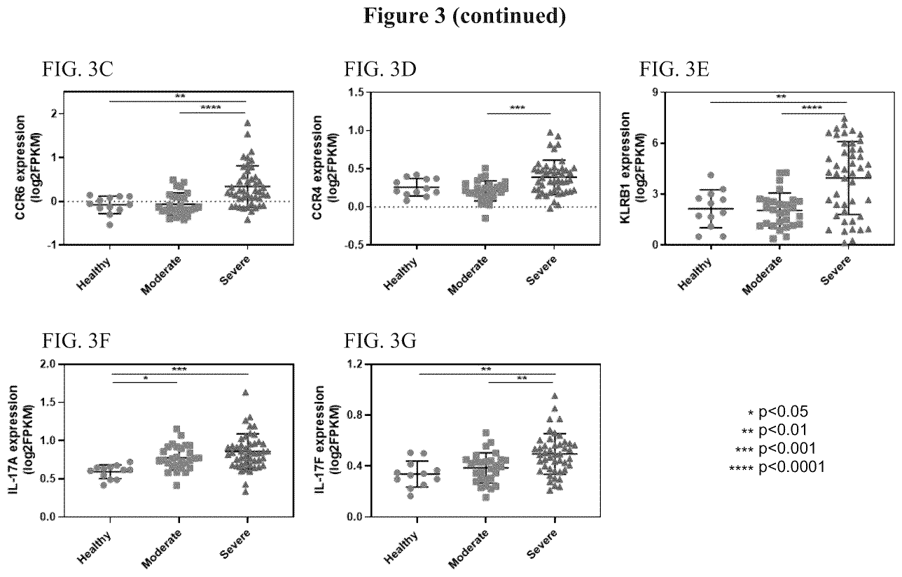

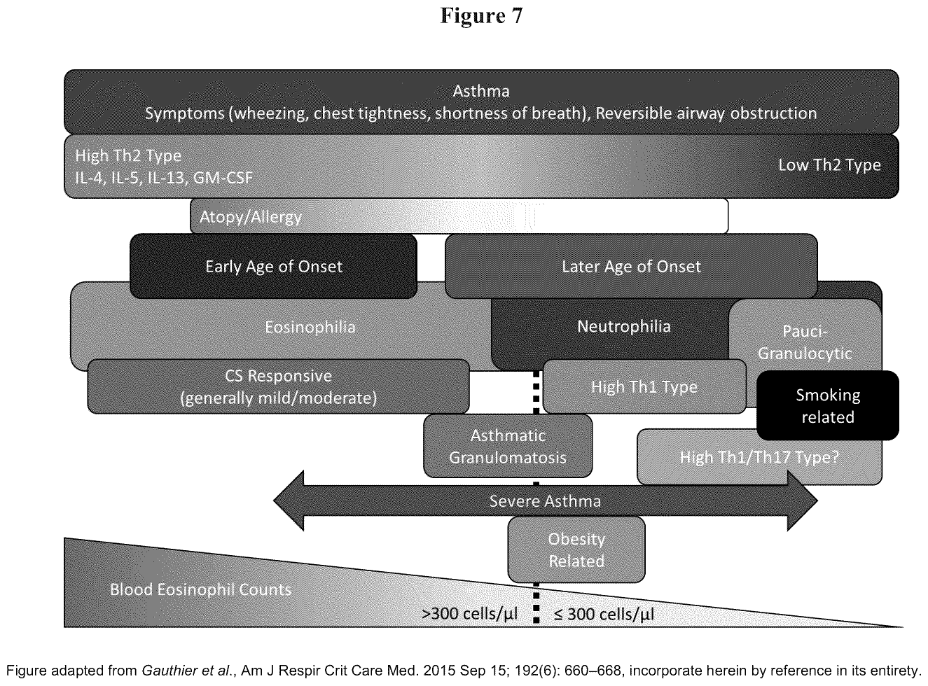

We report herein the surprising discovery that fatal severe asthma patients, who are steroid refractory, express significantly higher levels of CD6 protein and its ligand, ALCAM, in lung tissue than do patients without asthma (FIG. 2). Further, bioinformatics-based de novo analysis of publically available RNASeq datasets confirms this result by demonstrating that severe asthma patients express significantly higher levels of CD6 mRNA in comparison to moderate asthma patients and non-asthmatics in bronchiolar lavage fluid (BALF) (FIG. 3B). Moreover, bioinformatics-based de novo analysis of these datasets also demonstrated that Th17 markers and cytokines CCR6, CCR4, KLRB1, IL-17A, and IL-17F are all expressed significantly higher in severe asthma patients as compared to moderate asthma patients and non-asthmatics in bronchiolar lavage fluid (BALF) (FIGS. 3C-3G). Given the steroid refractory nature of the disease in these severe asthma patients (suggesting a non-Th2 disease, as Th2-mediated asthma typically responds well to steroid treatment, see FIG. 7), and the high expression of these Th17-markers and cytokines, we conclude that this elevated CD6 expression is due predominantly to Th17 T cells infiltrating the lung tissue. Interestingly, a recent report suggests that cosinophilic Th2-driven allergic

asthma may be mediated, in part, by ALCAM, as ALCAM knockout mice and mice treated with anti-ALCAM antibodies show reduced Th2 cytokine levels in response to allergen exposure. Kim, et al., Am J Respir Crit Care Med. 2018 Apr. 15;197 (8): 994-1008. However, these findings were limited to allergic eosinophilic Th2-mediated responses (focused on Th2 cytokines including IL-4, IL-5, IL-13, and IgE) in allergic asthma models; whereas, in contrast, our above-mentioned results focus on cases of non-allergic severe asthma, which exhibit low or no eosinophils. Further, our findings suggest that high levels of Th1 and Th17 T cells (expressing significantly elevated levels of Th17 markers (CCR6, CCR4, KLRB1, IL-17A, and IL-17F) may underlie the pathology in severe steroid refractory asthma. Interestingly, our data also differs from the findings reported in Kim, et al., in that they saw low resident ALCAM protein in the lung due to ALCAM shedding (via ADAM family metalloproteinase activity); whereas, in contrast, we observed high levels of ALCAM protein in the lungs of non-allergic severe asthma patients (FIG. 2). Thus, our data in light of the Kim et al., report, further illustrate the complex differences between eosinophilic and non-eosinophilic types of asthma, which likely underlie the generally steroid-responsive nature of the former and the generally steroid-refractory or resistant nature of the latter.

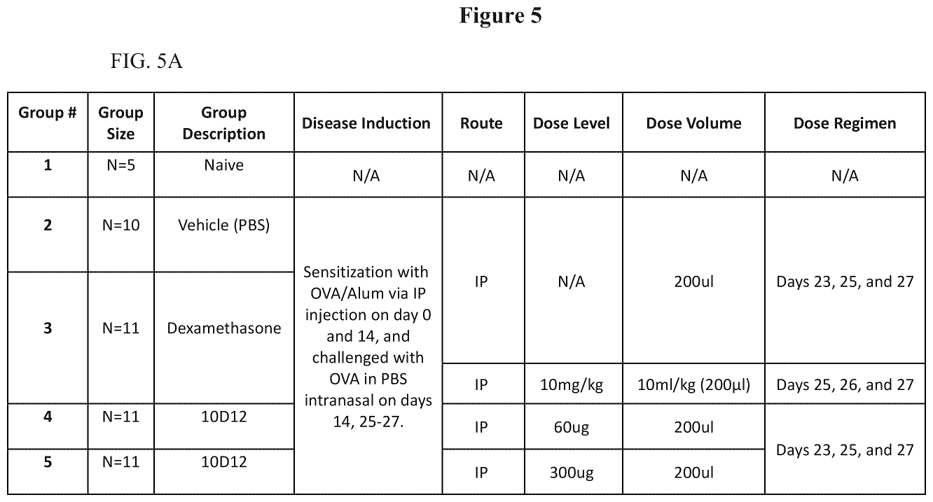

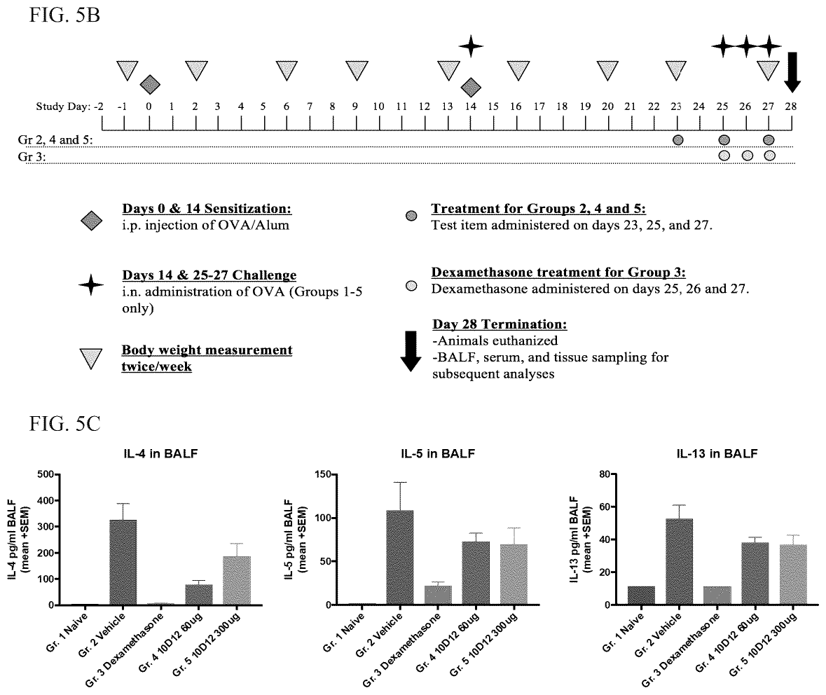

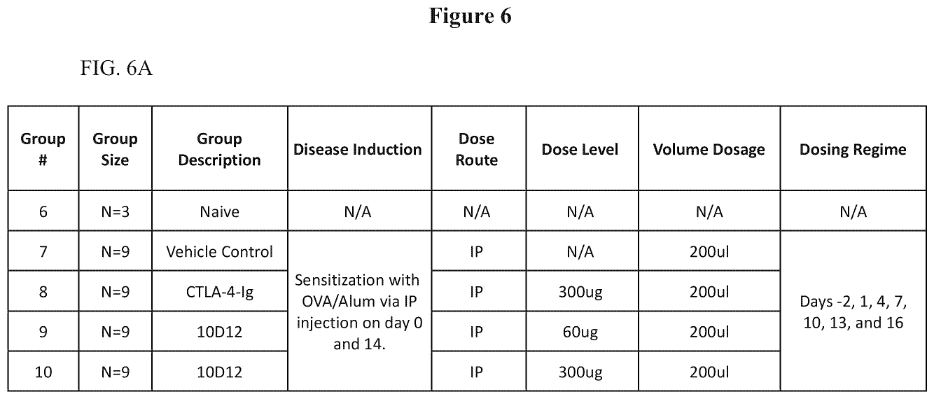

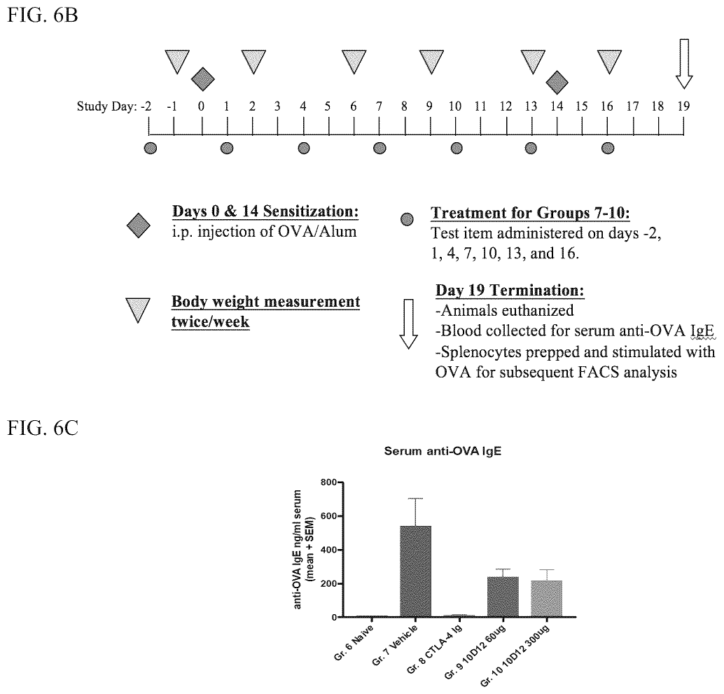

Notably, we also report herein that in a murine model of Th2-mediated allergic asthma (the same model utilized in Kim et al., above), CD6 inhibition effectively decreases Th2 cytokines in bronchiolar lavage fluid (FIG. 5C) and inhibits Th2-driven IgE production (FIG. 6), showing that eosinophilic Th2 asthma may also be treatable with an anti-CD6 antibody. Thus, unlike the bifurcated response to steroid treatment that is a nearly a hallmark difference between eosinophilic and non-eosinophilic types of asthma, our data suggests that CD6 inhibition might provide a one-stop mechanism for the concurrent inhibition of Th2-and Th1/Th17-mediated asthma. To our knowledge, this is the first report that suggests treating severe asthma characterized as having low or no eosinophilic component (i.e., Th1/Th 17-mediated asthma, rather than Th2-mediate allergic asthma) with a CD6 antibody.

Accordingly, certain aspects of the present disclosure provide methods and compositions directed to inhibiting T cell-mediated pulmonary inflammation in a subject that has asthma comprising inhibiting or blocking the CD6-signaling pathway. In particular embodiments, the methods and compositions are directed to inhibiting T cell-mediated pulmonary inflammation in a subject that has an asthma characterized by low or no blood eosinophils. Methods for determining blood eosinophil counts are well known in the art and may be used in accordance with the present disclosure (see, e.g., Kostikas, et al., Curr Drug Targets. 2018 December; 19 (16): 1882-1896, incorporated herein by reference in its entirety).

As will be clear to a person of ordinary skill in the art, the presence of high or low eosinophils is the well-excepted gold standard for differentiating allergic asthma from other forms of non-allergic asthma. Eosinophils normally represent less than 5% of the leukocytes in peripheral blood, but in response to type 2 helper T-cell (Th2)-mediated inflammation (such as is present in allergic asthma) their production increases greatly, and many clinical studies have used measurements of blood eosinophil counts as a surrogate for lung eosinophil levels. See, e.g., Kostikas, et al., Curr Drug Targets. 2018 December; 19 (16): 1882-1896, incorporated herein by reference in its entirety. As used herein, reference to an asthma subject with “low” eosinophils means that the subject has blood eosinophil counts ≤300 cells/μl. Reference to an asthma subject with “no” eosinophils means that the subject has no detectable blood eosinophil cells/μl.

Certain aspects of the present disclosure provide methods and compositions directed to inhibiting T cell-mediated pulmonary inflammation in a subject that has asthma comprising, administering to a subject an anti-CD6 antibody (e.g., EQ001), or an antigen binding fragment thereof. In some embodiments, the asthma may be severe asthma. In particular embodiments, the asthma may be characterized by low or no eosinophils. In particular embodiments, the asthma may be neutrophilic asthma. In particular embodiments, the asthma may be paucigranulocytic asthma. In particular embodiments, the asthma may be a mixed inflammation asthma.

Certain aspects of the present disclosure provide methods and compositions directed to treating, preventing, or attenuating the migration of a T cell into and through a pulmonary tissue in response to an asthma-inducing antigen comprising, administering to a subject an anti-CD6 antibody (e.g., EQ001), or an antigen binding fragment thereof. In some embodiments, the asthma may be severe asthma. In particular embodiments, the asthma may be characterized by low or no eosinophils. In particular embodiments, the asthma may be neutrophilic asthma. In particular embodiments, the asthma may be paucigranulocytic asthma. In particular embodiments, the asthma may be a mixed inflammation asthma.

Certain aspects of the present disclosure provide methods and compositions directed to modulating or attenuating a symptom or the severity of asthma comprising, administering to a subject an anti-CD6 antibody (e.g., EQ001), or an antigen binding fragment thereof. In some embodiments, the asthma may be severe asthma. In particular embodiments, the asthma may be characterized by low or no eosinophils. In particular embodiments, the asthma may be neutrophilic asthma. In particular embodiments, the asthma may be paucigranulocytic asthma. In particular embodiments, the asthma may be a mixed inflammation asthma.

Certain aspects of the present disclosure provide methods and compositions directed to modulating or attenuating a symptom or the severity of asthma comprising, contacting a T-cell with an anti-CD6 antibody (e.g., EQ001), or an antigen binding fragment thereof. In some embodiments, the asthma may be severe asthma. In particular embodiments, the asthma may be characterized by low or no eosinophils. In particular embodiments, the asthma may be neutrophilic asthma. In particular embodiments, the asthma may be paucigranulocytic asthma. In particular embodiments, the asthma may be a mixed inflammation asthma.

Certain aspects of the present disclosure provide methods and compositions directed to inhibiting T cell-mediated pulmonary inflammation in a subject that has asthma comprising, administering to a subject a binding partner that binds specifically to CD6 on a T cell and prevents or inhibits activation of CD6 signaling. The binding partner may be an anti-CD6 antibody (e.g., EQ001), or an antigen binding fragment thereof. In some embodiments, the asthma may be severe asthma. In particular embodiments, the asthma may be characterized by low or no eosinophils. In particular embodiments, the asthma may be neutrophilic asthma. In particular embodiments, the asthma may be paucigranulocytic asthma. In particular embodiments, the asthma may be a mixed inflammation asthma.

Certain aspects of the present disclosure provide methods and compositions directed to treating, preventing, or attenuating the migration of a T cell into and through a pulmonary tissue in response to an asthma-inducing antigen comprising, administering to a subject a binding partner that binds specifically to CD6 on a T cell and prevents or inhibits activation of CD6 signaling. The binding partner may be an anti-CD6 antibody (e.g., EQ001), or an antigen binding fragment thereof. In some embodiments, the asthma may be severe asthma. In particular embodiments, the asthma may be characterized by low or no eosinophils. In particular embodiments, the asthma may be neutrophilic asthma. In particular embodiments, the asthma may be paucigranulocytic asthma. In particular embodiments, the asthma may be a mixed inflammation asthma.

Certain aspects of the present disclosure provide methods and compositions directed to modulating or attenuating a symptom or the severity of asthma comprising, administering to a subject a binding partner that binds specifically to CD6 on a T cell and prevents or inhibits activation of CD6 signaling. The binding partner may be an anti-CD6 antibody (e.g., EQ001), or an antigen binding fragment thereof. In some embodiments, the asthma may be severe asthma. In particular embodiments, the asthma may be characterized by low or no eosinophils. In particular embodiments, the asthma may be neutrophilic asthma. In particular embodiments, the asthma may be paucigranulocytic asthma. In particular embodiments, the asthma may be a mixed inflammation asthma.

Certain aspects of the present disclosure provide methods and compositions directed to modulating or attenuating a symptom or the severity of severe asthma comprising, contacting a T-cell with a binding partner that binds specifically to CD6 on a T cell and prevents or inhibits activation of CD6 signaling. The binding partner may be an anti-CD6 antibody (e.g., EQ001), or an antigen binding fragment thereof. In some embodiments, the asthma may be severe asthma. In particular embodiments, the asthma may be characterized by low or no eosinophils. In particular embodiments, the asthma may be neutrophilic asthma. In particular embodiments, the asthma may be paucigranulocytic asthma. In particular embodiments, the asthma may be a mixed inflammation asthma.

Certain aspects of the present disclosure provide methods and compositions directed to treating, preventing, or attenuating the migration of a T cell into and through a pulmonary tissue in response to an asthma-inducing antigen comprising inhibiting or blocking the CD6 pathway. In some embodiments, the asthma may be severe asthma. In particular embodiments, the asthma may be characterized by low or no eosinophils. In particular embodiments, the asthma may be neutrophilic asthma. In particular embodiments, the asthma may be paucigranulocytic asthma. In particular embodiments, the asthma may be a mixed inflammation asthma.

Certain aspects of the present disclosure provide methods and compositions directed to modulating or attenuating a symptom or the severity of asthma comprising inhibiting or blocking the CD6 pathway. In some embodiments, the asthma may be severe asthma. In particular embodiments, the asthma may be characterized by low or no eosinophils. In particular embodiments, the asthma may be neutrophilic asthma. In particular embodiments, the asthma may be paucigranulocytic asthma. In particular embodiments, the asthma may be a mixed inflammation asthma.

Certain aspects of the present disclosure provide methods and compositions directed to modulating or attenuating a symptom or the severity of asthma comprising inhibiting or blocking the CD6 pathway. In some embodiments, the asthma may be severe asthma. In particular embodiments, the asthma may be characterized by low or no eosinophils. In particular embodiments, the asthma may be neutrophilic asthma. In particular embodiments, the asthma may be paucigranulocytic asthma. In particular embodiments, the asthma may be a mixed inflammation asthma.

In some embodiments, the method of treating severe asthma comprises modulating the activation, proliferation, differentiation, survival and/or migration of one or more CD6-expressing cells is by contacting the cell with an anti-CD6 antibody (e.g., EQ001). For example, the CD6-expressing cell that is contacted with an anti-CD6 antibody (e.g., EQ001) may be a T cell that expresses CD6. Accordingly, such properties of such a T cell may be modulated in a subject that has severe asthma via the administration of an anti-CD6 antibody. In some embodiments the T cell that is so modulated is a CD4+ T cell. In some embodiments the T cell that is so modulated is a T-helper 1 (Th1) T cell. In some embodiments the T cell that is so modulated is a T-helper 17 (Th17) T lymphocyte (T cell). In some embodiments, the anti-CD6 antibody (e.g., EQ001) modulates the activation, differentiation, survival and/or migration of a Th1 T cell and/or a Th17 T cell as well as another cell expressing CD6. For example, the anti-CD6 antibody (e.g., EQ001) may modulate the activation, differentiation, survival and/or migration of a combination of cells selected from (i) a Th17 T cell and a Th2 T cell; (ii) a T17 T cell and a Th1 T cell; (iii) Th1 T cell and a Th2 T cell; and (iv) a Th17 T cell, a Th1 T cell, and a Th2 T cell. In some embodiments, the asthma may be severe asthma. In particular embodiments, the asthma may be characterized by low or no cosinophils. In particular embodiments, the asthma may be neutrophilic asthma. In particular embodiments, the asthma may be paucigranulocytic asthma. In particular embodiments, the asthma may be a mixed inflammation asthma.

In some embodiments, the asthma is a severe asthma that includes neutrophilic, or a mixed-phenotype that is both cosinophilic and neutrophilic. In some embodiments, the asthma is a severe asthma that includes neutrophilic, paucigranulocytic, or a mixed-phenotype that is both eosinophilic and neutrophilic. In some embodiments, the asthma is a neutrophilic severe asthma characterized by inflammation of the airways involving a CD6⁺ T cell that is a Th17 T cell. In some embodiments, the asthma is a neutrophilic severe asthma characterized by inflammation of the airways involving a CD6⁺ T cell that is Th1 T cell. In certain particular embodiments, the asthma is a neutrophilic severe asthma characterized by inflammation of the airways involving a Th1 CD6⁺ T cell and a Th17 CD6⁺ T cell. In some embodiments, the asthma is a paucigranulocytic severe asthma characterized by inflammation of the airways involving a CD6⁺ T cell that is a Th17 T cell. In some embodiments, the asthma is a paucigranulocytic severe asthma characterized by inflammation of the airways involving a CD6⁺ T cell that is Th1 T cell. In certain particular embodiments, the asthma is a paucigranulocytic severe asthma characterized by inflammation of the airways involving a Th1 CD6⁺ T cell and a Th17 CD6⁺ T cell. In some embodiments, the severe asthma is a mixed neutrophilic and eosinophilic and asthma characterized by inflammation of the airways involving a CD6⁺ T cell that is a Th1 T cell a CD6⁺ T cell that is a Th17 T cell, and a CD6⁺ T cell that is a Th2 T cell.

In particular embodiments, the present disclosure provides a method comprising administering an anti-CD6 antibody (e.g., EQ001) to a patient that has a neutrophilic severe asthma characterized by inflammation of the airways involving a CD6⁺ T cell that is a Th17 T cell.

In particular embodiments, the present disclosure provides a method comprising administering an anti-CD6 antibody (e.g., EQ001) to a patient that has a neutrophilic severe asthma characterized by inflammation of the airways involving a CD6⁺ T cell that is a Th1 T cell.

In particular embodiments, the present disclosure provides a method comprising administering an anti-CD6 antibody (e.g., EQ001) to a patient that has a neutrophilic severe asthma characterized by inflammation of the airways involving a CD6⁺ T cell that is a Th17 T cell and a CD6⁺ T cell that is a Th1 T cell.

In certain embodiments, the present invention relates to treating a severe asthma that comprises low eosinophilic T cell response with an anti-CD6 antibody disclosed herein (e.g., EQ001). In certain embodiments, the present invention relates to treating a severe asthma that comprises no eosinophilic T cell response with an anti-CD6 antibody disclosed herein (e.g., EQ001).

In certain embodiments, the present invention relates to treating a severe asthma that comprises a neutrophilic T cell response, but no eosinophilic T cell response with an anti-CD6 antibody disclosed herein (e.g., EQ001). In certain embodiments, the present invention relates to treating a severe asthma that comprises a neutrophilic T cell response, but substantially no eosinophilic T cell response with an anti-CD6 antibody disclosed herein (e.g., EQ001). In certain embodiments, the present invention relates to treating a severe asthma that comprises a neutrophilic T cell response and a low eosinophilic T cell response with an anti-CD6 antibody disclosed herein (e.g., EQ001). In certain embodiments, the present invention relates to treating a severe asthma that comprises a T cell response that is substantially Th17 CD4⁺ cell mediated with an anti-CD6 antibody disclosed herein (e.g., EQ001). In certain embodiments, the present invention relates to treating a severe asthma that comprises a T cell response that is substantially mediated by Th1 and Th17 CD4⁺ effector cells with an anti-CD6 antibody disclosed herein (e.g., EQ001). In certain embodiments, the present invention relates to treating an asthma (e.g., a severe asthma) that comprises a T cell response that is mediated by at least 2 fold (2×) more Th1 and/or Th17 CD4³⁰ effector cells than Th2 CD4⁺ effector cells, or at 3×, 4×, 5×, 10×, 15×, 20×, 30×, 40×, 50×, 100×, 1,000×, 10,000× more Th1 and/or Th17 CD4⁺ effector cells than Th2 CD4⁺ effector cells.

The binding partner that binds specifically to CD6 on a T cell and prevents or inhibits activation of CD6 signaling may be an anti-CD6 antibody or an antigen binding portion thereof.

The anti-CD6 antibody may be any antibody that binds to CD6 and blocks CD6-mediated downstream signaling in a T cell. For example, blocking studies using anti-CD6 monoclonal antibodies (mAbs) suggest that CD6 plays an important role in T cell development by regulating T cell adhesive interactions with thymic epithelial (TE) cells (Patel et al., J. Exp. Med. (1995) 181:1563-1568). Additional studies have shown that CD6 can function as an important accessory molecule in T cell activation. For example, certain anti-CD6 mAb are directly mitogenic for T cells (Gangemi et al., J. Immunol. (1989) 143:2439; Bott et al., Int. Immunol. (1993) 7:783), whereas others are able to co-stimulate T cell proliferation in conjunction with anti-CD3, anti-CD2 or PMA (Gangemi et al., J. Immunol. (1989) 143:2439; Morimoto et al., J. Immunol. (1988) 140:2165-2170; Osorio et al., Cell. Immunol. (1994) 154:23). Yet additional evidence of the role of CD6 in T cell activation comes from studies showing that CD6 becomes hyperphosphorylated on Ser and Thr residues (Swack et al., Mol. Immunol. (1989) 26:1037-1049; Swack et al., J. Biol. Chem. (1991) 266:7137; Cardenas et al., J. Immunol., 145:1450-1455 (1990)) and phosphorylated on Tyr residues (Wee et al., J. Exp. Med. (1993) 177:219-223) following T cell activation. These and other studies implicate CD6 as an important modulator of both immature and mature T cell function in vivo, affecting both T cell activation and signal transduction (De Wit, J., et al., Blood (2011) 118:6107-6114), and any antibody that is able to prevent these effects is suitable for use in the present invention.

U.S. Pat. No. 6,372,215 discloses antibodies and other binding agents that bind specifically to SRCR domains 3 (D3) of human CD6 (hCD6) or human CD6 stalk domain (CD6S) and inhibit activated leukocyte cell adhesion molecule (ALCAM) binding to CD6.

Earlier publications and patents disclosed sequences of the murine anti-CD6 (IOR-T1) monoclonal antibody and the amino acid modifications that were carried out to humanize IOR-TI to T1h (humanized IOR-T1). U.S. Pat. No. 5,712,120 and its equivalent EP 0699755 disclose specific methods to humanize murine monoclonal antibodies and the sequence of IOR-TI and T1h. U.S. Pat. No. 6,572,857 and its equivalent EP 0807125 disclose the sequence of IOR-TI and T1h (humanized IOR-T1). PCT/IN2008/00562, and related U.S. Pat. No. 8,524,233, entitled “A Monoclonal Antibody and a Method Thereof,” disclose the production of an anti-CD6 antibody in NSO cells, which has the heavy and light chain sequences provided herein as SEQ ID NOS: 1 and 2. This antibody was also referred to in that patent as T1h, although its sequence differed from the original T1h. The INN name for this antibody is itolizumab. Itolizumab is produced in the mouse derived NSO cell line and in Chinese Hamster Ovary (CHO) cells, and is referred to herein by its trade name EQ001, when produced in CHO cells and by its trade name ALZUMAb, when produced in NSO cells. EQ001 (i.e., itolizumab produced in CHO cells) is also known in the art as “Bmab-600.” In various embodiments herein, we refer to the antibody itself, irrespective of its production method, by its INN name, itolizumab. Thus, the term itolizumab, as used herein, encompasses ALZUMAb and EQ001, each of which have the same sequence as itolizumab. The amino acid sequences of the variable heavy (VH) and variable light (VK) of itolizumab (and EQ001/ALZUMAb) are provided herein as SEQ ID NOS: 1 and 2, respectively. The nucleotide (DNA) sequences of the VH and VK of itolizumab (and EQ001/ALZUMAb) are provided herein as SEQ ID NOS: 3 and 4, respectively. The amino acid sequence of the itolizumab (and EQ001/ALZUMAb) VH CDRs 1-3 are provided as SEQ ID NOS: 5-7, respectively. The amino acid sequence of the itolizumab (and EQ001/ALZUMAb) VK CDRs 1-3 are provided as SEQ ID NOS: 8-10, respectively.

Accordingly, the anti-CD6 antibody may be an anti-CD6 monoclonal antibody that comprises a heavy chain and light chain variable region comprising an amino acid sequence as set forth in SEQ ID NO: 1 and SEQ ID NO: 2.

The anti-CD6 antibody may be an anti-CD6 monoclonal antibody that comprises a heavy chain and light chain variable region comprising the nucleotide sequence set forth in SEQ ID NO: 3 or a complement thereof; and (b) a nucleic acid molecule comprising the nucleotide sequence set forth in SEQ ID NO: 4 or a complement thereof.

The anti-CD6 antibody may be an anti-CD6 monoclonal antibody that comprises a heavy chain and light chain variable region comprising an amino acid sequence which is at least 80% homologous to the amino acid sequence as set forth in SEQ ID NO: 1 and SEQ ID NO: 2.

The anti-CD6 antibody may be an anti-CD6 monoclonal antibody that specifically binds CD6 and comprises at least about 65% amino acid sequence identity or homology, at least about 70% amino acid sequence identity or homology, at least about 75% amino acid sequence identity or homology, at least about 80% amino acid sequence identity or homology, at least about 80% amino acid sequence identity or homology, at least about 85% amino acid sequence identity or homology, at least about 90% amino acid sequence identity or homology, at least about 95% amino acid sequence identity or homology, at least about 98% amino acid sequence identity or at least about 99% amino acid sequence identity or homology in that portion corresponding to amino acid residues represented by the SEQ ID Nos 1 & 2.

The anti-CD6 antibody may comprise one or more CDRs selected from EQ001 heavy chain CDR1: GFKFSRYAMS (SEQ ID NO: 5); EQ001 heavy chain CDR2: TISSGGSYIYYPDSVKG (SEQ ID NO: 6); EQ001 heavy chain CDR3: RDYDLDYFDS (SEQ ID NO: 7); EQ001 light chain CDR1: KASRDIRSYLT (SEQ ID NO: 8); EQ001 light chain CDR2: YATSLAD (SEQ ID NO: 9); EQ001 light chain CDR3: LQHGESP (SEQ ID NO: 10); and combinations thereof.

In particular embodiments, the anti-CD6 antibody comprises each of the EQ001 CDRs provided as SEQ ID NOS: 5-10. In particular embodiments, the anti-CD6 antibody is a humanized antibody that comprises each of the EQ001 CDRs provided as SEQ ID NOS: 5-10. In particular embodiments, the anti-CD6 antibody is a humanized IgG antibody that comprises each of the EQ001 CDRs provided as SEQ ID NOS: 5-10. In particular embodiments, the anti-CD6 antibody is a humanized IgG1 antibody that comprises each of the EQ001 CDRs provided as SEQ ID NOS: 5-10. In particular embodiments, the anti-CD6 antibody is a humanized antibody produced in a CHO cell, wherein the humanized antibody comprises each of the EQ001 CDRs provided as SEQ ID NOS: 5-10.

The anti-CD6 antibody may be selected from UMCD6 mAb (Li et al., PNAS Mar. 7, 2017, vol. 114, no. 10, 2687-2692, incorporated herein by reference in its entirety) and any one of the antibodies listed on Table 2:

TABLE 2
Anti-CD6 antibodies

| Name | Specificity | Isotype | Notes | References |
|---|---|---|---|---|
| OX126 | Human CD6 | Mouse IgG1 | Raised against recombinant CD6 domain 3 | [5] |
| | domain [symbol] | | and [symbol] with rat CD6. Blocks soluble CD166 | |
| | | | binding to CD6. Blocks interactions | |
| | | | between cells | |
| T12 | Human CD6 | mouse IgM | Blocks interactions between cells. Used clinically | [12] and |
| | domain 1 | | [symbol] T cells | reviewed |
| | | | | in [51] |
| UMCD6 | Human CD6 | Mouse IgG1 | Higher affinity for CD6 than MT605. Competes with | [57] |
| | domain 1 | | IOR-T [symbol] . Blocks interactions between cells. | |
| MT [symbol] | Human CD6 | Mouse IgG1 | Lower affinity for recombinant CD6 [symbol] UMCD6. | [57] |
| | domain 1 | | Partially competes with IOR-T [symbol] . Blocks | |
| | | | interactions betweeen cells. | |
| IOR- | Human CD6 | Mouse [symbol] /human | Competes with UMCD6 and partially with MT605. | [57] |
| T1/T1h | domain 1 | IgG1 | Blocks [symbol] between cells. Used clinically | |
| | | | as [symbol] immunosuppressant | |
| 34-81 | Human CD166 [symbol] | Mouse IgG1 | Partially inhibits soluble CD6 binding to cells. | [ [symbol] .29.39] |
| | Bound [symbol] but | | Can both promote and inhibit CD166 [symbol] | |
| | not V [symbol] or VCCC. | | interactions [symbol] depending on valency. | |
| AZN- | Human CD166 | Mouse IgG [symbol] | Inhibits CD166 [symbol] interactions | [29] |
| [symbol] | C2 [symbol] | | proposed to be by inhibiting CD166 [symbol] | |
| MAB656 | Human CD166 | Mouse IGg1 | [symbol] effects reported in humans and mice | [59]. Lee I. |
| | VCCC | | but dose not bind to recombinant mouse CD166. | Garner, unpublished |
| | | | | data |
| [symbol] | Human CD166 | Human single | Blocks CD6/CD166 and CD166/CD166 interactions | [60] |
| | domain 1 | chain antibody | and binds monkey and murine CD166. | |
| | | fragment | | |

[symbol]

indicates data missing or illegible when filed

The anti-CD6 antibody may be T1h as disclosed in US Pat. No. 8,524,233, incorporated herein by reference in its entirety.

The anti-CD6 antibody may be itolizumab. The anti-CD6 antibody may be ALZUMAb. The anti-CD6 antibody may be EQ001.

The anti-CD6 antibody may be an antibody produced by secreting hybridoma IOR-TIA deposited with the ECACC as deposit No. ECACC 96112640.

The anti-CD6 antibody may bind to CD6 on the surface of a T cell. The anti-

CD6 antibody may bind to domain 1, domain 2, or domain 3 of CD6 on the surface of a T cell. In certain aspects the anti-CD6 antibody binds to domain 1 or domain 3 on CD6. In particular embodiments, the anti-CD6 antibody binds to domain 3 on CD6. The binding of the anti-CD6 antibody to the CD6 on the surface of the T cell may modulate the activity of the T cell. In certain aspects, the binding of the anti-CD6 antibody to CD6 on the surface of a T cell modulates the activity and/or migration of the T cell. In particular aspects, the binding of the anti-CD6 antibody to CD6 on the surface of a T cell modulates migration of the T cell into and through a lung tissue.

The anti-CD6 antibody (e.g., EQ001) may be delivered to the subject as an anti-CD6 pharmaceutical composition.

Pharmaceutical compositions suitable for the delivery of an anti-CD6 antibody and methods for their preparation will be readily apparent to those skilled in the art. Such compositions and methods for their preparation may be found, e.g., in Remington's Pharmaceutical Sciences, 19th Edition (Mack Publishing Company, 1995), incorporated herein by reference in its entirety. Pharmaceutical compositions containing anti-CD6antibodies are also known in the art. For example, the anti-CD6 antibody may be a pharmaceutical composition disclosed in U.S. patent application Ser. No. 12/525,449 (US20100047242), incorporated herein by reference in its entirety.

Pharmaceutical compositions of the present invention may comprise an active pharmaceutical agent (e.g., an anti-CD6 antibody such as EQ001) and one or more pharmaceutically acceptable carrier, excipients, diluent, surfactant, and/or vehicles.

The pharmaceutical composition may comprise an anti-CD6 antibody (or an antigen-binding fragment thereof) and one or more agent selected from the group consisting of carriers, excipients, diluents, antioxidants, preservatives, coloring, flavoring and diluting agents, emulsifying agents, suspending agents, solvents, fillers, bulking agents, buffers, delivery vehicles, tonicity agents, cosolvents, wetting agents, complexing agents, buffering agents, antimicrobials, and/or surfactants. Such agents are known in the art (see, e.g., Remington's Pharmaceutical Sciences, 18th edition, Mack Publishing Co., Easton, PA (1990), incorporated herein by reference in its entirety.

The present invention also includes combination therapies comprising administering to a patient an anti-CD6 antibody (e.g., EQ001), or an antigen binding portion thereof in combination with a second active agent, or a device or a procedure capable of treating, preventing, or attenuating one or more asthma related symptom. In this context “administered in combination” means: (1) part of the same unitary dosage form; (2) administration separately, but as part of the same therapeutic treatment program or regimen, typically but not necessarily, on the same day.

As previously noted, EQ001 (or another anti-CD6 antibody) may be administered alone as a monotherapy in some aspects or as a combination therapy in some aspects. In some aspects, any one of the EQ001 (or another anti-CD6 antibodies) described herein (e.g., EQ001) is for administering to a patient according to the methods disclosed herein may be administered in combination with one or more other therapeutic agent as a combination therapy. For example, a EQ001 (or another anti-CD6 antibody) may be administered to a patient as a combination therapy with another agent for the treatment of an inflammatory or autoimmune disease. The combination therapy may comprise administration of EQ001 (or another anti-CD6 antibody) an agent selected from, e.g., but not limited to, a steroid or an immunosuppressant. The steroid may be a corticosteroid. The corticosteroid may be prednisone.

EQ001 (or another anti-CD6 antibody) may be administered before, after, or concurrently with one or more of such anti-inflammatory or autoimmune disease agents. In some embodiments, such combinations may offer significant advantages, including additive or synergistic activity in therapy.

In various embodiments, the compositions and methods disclosed herein, e.g., the methods for treating asthma, involve administering to a subject an effective amount of EQ001 (or another anti-CD6 antibody) or a composition (e.g., a pharmaceutical composition) comprising a EQ001 (or another anti-CD6 antibody).

EQ001 (or another anti-CD6 antibody) may be administered as a pharmaceutical composition. The CD6-ALCAM pathway inhibitor may be administered before, after, and/or concurrently with the one or more other therapeutic. If administered concurrently with the one or more other therapeutic agent, such administration may be simultaneous (e.g., in a single composition) or may be via two or more separate compositions, optionally via the same or different modes of administration (e.g., local, systemic, oral, intravenous, etc.).

Administration of EQ001 (or another anti-CD6 antibody) and/or other therapeutic agents can be accomplished via any mode of administration for therapeutic agents. These modes include systemic or local administration such as oral, nasal, parenteral, transdermal, subcutaneous, vaginal, buccal, rectal or topical administration modes.

For administration in the methods of use described herein, EQ001 (or another anti-CD6 antibody) may be mixed, prior to administration, with a non-toxic, pharmaceutically acceptable carrier substance (e.g. normal saline or phosphate-buffered saline), and will be administered using any medically appropriate procedure, e.g., parenteral administration (e.g., injection) such as by intravenous or intra-arterial injection.

Formulations of EQ001 (or another anti-CD6 antibody) used in accordance with the present invention may be prepared by mixing an antibody having the desired degree of purity with optional pharmaceutically acceptable carriers, excipients or stabilizers in either the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives such as octadecyl dimethyl benzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol and m-cresol; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™, or polyethylene glycol (PEG).

EQ001 (or another anti-CD6 antibody) may also be entrapped in microcapsules prepared, for example, by coacervation techniques or by interfacial polymerization, for example, hydroxy methyl cellulose or gelatin-microcapsules and poly-(methyl methacrylate) microcapsules, respectively, in colloidal drug delivery systems (for example, liposomes, albumin microspheres, microemulsions, nano-particles and nanocapsules) or in macroemulsions. Such techniques are well known in the art.

Sustained-release preparations may be prepared. Suitable examples of sustained-release preparations include semipermeable matrices of solid hydrophobic polymers containing EQ001 (or another anti-CD6 antibody), which matrices are in the form of shaped articles, e.g. films, or microcapsules. Examples of sustained-release matrices include polyesters, hydrogels, copolymers of L-glutamic acid, non-degradable ethylene-vinyl acetate and degradable lactic acid-glycolic acid copolymers.

EQ001 (or another anti-CD6 antibody) may be administered to a subject in accord with known methods, such as intravenous administration as a bolus or by continuous infusion over a period of time, by intramuscular, intraperitoneal, intracerobrospinal, subcutaneous, intra-articular, intrasynovial, intrathecal or oral routes. In some instances, intravenous or subcutaneous administration of EQ001 (or another anti-CD6 antibody), is preferred.

Depending on the intended mode of administration, the disclosed compounds or pharmaceutical compositions can be in solid, semi-solid or liquid dosage form, such as, for example, injectables, tablets, suppositories, pills, time-release capsules, elixirs, tinctures, emulsions, syrups, powders, liquids, suspensions, or the like, sometimes in unit dosages and consistent with conventional pharmaceutical practices. Likewise, they can also be administered in intravenous (both bolus and infusion), intraperitoneal, subcutaneous or intramuscular form, and all using forms well known to those skilled in the pharmaceutical arts. Pharmaceutical compositions suitable for the delivery of EQ001 (or another anti-CD6 antibody) (alone or, e.g., in combination with another therapeutic agent according to the present disclosure) and methods for their preparation will be readily apparent to those skilled in the art. Such compositions and methods for their preparation may be found, e.g., in Remington's Pharmaceutical Sciences, 19th Edition (Mack Publishing Company, 1995), incorporated herein in its entirety.

The dosage regimen utilizing EQ001 (or another anti-CD6 antibody) is selected in accordance with a variety of factors including type, species, age, weight, sex and medical condition of the patient; the severity of the condition to be treated; the route of administration; the renal or hepatic function of the patient; and the particular disclosed compound employed. A physician or veterinarian of ordinary skill in the art can readily determine and prescribe the effective amount of the drug required to prevent, counter or arrest the progress of the condition.

An exemplary, non-limiting range for a therapeutically effective amount of EQ001 (or another anti-CD6 antibody) used in the present invention is about 0.01-100 mg/kg per subject body weight, such as about 0.01-50 mg/kg, for example about 0.01-25 mg/kg. A medical professional having ordinary skill in the art may readily determine and prescribe the effective amount of the pharmaceutical composition required. For example, a physician could start doses of the EQ001 (or another anti-CD6 antibody) at levels lower than that required in order to achieve the desired therapeutic effect and gradually increase the dosage until the desi red effect is achieved.

In one embodiment EQ001 (or another anti-CD6 antibody) is administered by infusion in a weekly dosage of from 1 to 500 mg kg per subject body weight such as, from 20 to 200 mg/kg. Such administration may be repeated, e.g., 1 to 8 times, such as 3 to 5 times. In the alternative, the administration may be performed by continuous infusion over a period of from 2 to 24 hours, such as, from 2 to 12 hours.

In one embodiment EQ001 (or another anti-CD6 antibody) is administered in a weekly dosage of from 0 mg to 200 mg, for up to 7 times, such as from 4 to 6 times. The administration may be performed by continuous infusion over a period of from 2 to 24 hours, such as, from 2 to 12 hours. Such regimen may be repeated one or more times as necessary, for example, after 6 months or 2 months.

In some aspects of these combination therapies, the second active agent is one or more agent capable of modulating the immune system. In some aspects of these combination therapies, the second active agent is one or more immunosuppressant. In some aspects of these combination therapies, the second active agent is one or more beta agonist.

In some aspects of these combination therapies, the second active agent is one or more short acting beta agonist. In some aspects of these combination therapies, the second active agent is albuterol. In some aspects, the albuterol is administered in a dosage form selected from: an aerosol powder; a solution; a capsule; and a powder suspension. In some aspects of these combination therapies, the second active agent is a steroid, e.g., a corticosteroid. In some aspects, the corticosteroid is administered in a dosage form selected from a tablet, a delayed release capsule; an extended release tablet; an extended release capsule; a syrup; a solution; an elixir; a suspension; a delayed release tablet; a liquid; and a disintegrating tablet.

In some aspects of these combination therapies, the second active agent is Ipratropium. In some aspects, the Ipratropium is administered in a spray dosage form.

In some aspects of these combination therapies, the second active agent comprises one or more agent selected from beclomethasone dipropionate; budesonide; flunisolide; fluticasone propionate; mometasone furoate; triamcinolone acetonide; dexamethasone; hydrocortisone; methylprednisone; prednisolone; prednisone; formoterol fumarate; salmeterol xinafoate; albuterol sulfate; isoetharine hydrochloride; isoproterenol hydrochloride; levalbuterol hydrochloride; pirbuterol acetate; terbutaline sulfate; ipratropium bromide; montelukast sodium; zafirlukast; zileuton; oxytriphylline; theophylline; cromolyn sodium; nedocromil sodium; omalizumab; a combined medication comprising fluticasone and salmeterol; a combined medication comprising xinafoate (inhaled steroid plus a long-acting beta-2 agonist); a combined medication comprising budesonide and formoterol fumarate (inhaled steroid plus long-acting beta-2 agonist); a combined medication comprising fluticasone propionate and salmeterol xinafoate (inhaled steroid plus long-acting beta-2 agonist); a combined medication comprising budesonide and formoterol fumarate (inhaled steroid plus long-acting beta-2 agonist); a combined medication comprising ipratropium bromide and albuterol sulfate; or combinations thereof.

In some aspects, the present invention also includes combination therapies comprising administering to a patient an anti-CD6 antibody (e.g., EQ001), or an antigen binding portion thereof in combination with a procedure selected from intubation, mechanical ventilation, oxygen therapy, and combinations thereof. Such procedures may also be administered to the subject in combination with any one or more of the aforementioned additional agents that are useful in the combination therapies described herein.

In certain aspects, the methods disclosed herein, which may include administering an anti-CD6 antibody (e.g., EQ001), or an antigen binding portion thereof), to a subject, may provide treatment of one or more asthma related symptom.

Optimal dosages and dosage regimens to be administered may be readily determined by those skilled in the art and will vary with the pharmacodynamic characteristics of the particular agent, its time and mode of administration, the strength of the preparation and the advancement of the disease condition (including the nature and extent of the symptoms of the disease). In addition, factors associated with the particular patient being treated, including patient's sex, age, weight, diet, physical activity and concomitant diseases, will result in the need to adjust dosages and/or regimens.

All of the U.S. patents, U.S. patent application publications, U.S. patent applications, PCT patent application, PCT patent application publications, foreign patents, foreign patent applications and non-patent publications referred to in this specification or listed in any Application Data Sheet are incorporated herein by reference in their entirety. From the foregoing it will be appreciated that, although specific embodiments of the invention have been described herein for purposes of illustration, various modifications may be made without deviating from the spirit and scope of the invention.

### EXAMPLES

### Example 1

### CD6 Expression is Significantly Elevated in Human Fatal Asthma Patients

Summary of Results: Lung tissue taken from fatal asthma patients exhibit high infiltration of CD6⁺ cells into the lamina propria which overexpresses ALCAM in this region compared to controls.

Lungs were obtained fresh from either fatal asthma patients or non-asthmatic patients. Tissue samples were fixed and subsequently stained for CD6, ALCAM expression by immunofluorescence. ALCAM expression in the lamina propria was elevated in the fatal asthma patients; whereas ALCAM expression was absent in the lamina propria of non-asthma controls. The region of ALCAM staining localized with high levels of infiltrating CD6⁺ cells in these lungs (FIG. 2). This suggests that severe asthma is associated with infiltration by CD6⁺ T cells and that ALCAM expression may be involved in the migration/infiltration of the CD6⁺ cells into the lung during severe/fatal asthma. In contrast, prior work has demonstrated that ALCAM is decreased in the lung tissues of animals exposed to allergens in allergic asthma models, due to increased metalloprotease-mediated ALCAM shedding.

### Example 2

### CD6 Expression is Significantly Elevated in Human Fatal Asthma Patients

Summary of Results: De novo analysis of a publicly available RNASeq dataset confirms that severe asthma patients express significantly higher levels of CD6 in comparison to moderate asthma patients and non-asthmatics.

RNA data was obtained from cell pellets collected by bronchiolar lavage as a part of two longitudinal prospective clinical studies:
BOBCAT study (Arron et al., Eur Respir J. 2014 February;43 (2): 627-9)
MAST study (Simpson et al., Nat Immunol. 2014 December;15 (12): 1162-70)

The transcriptome of these samples was determined by RNASeq and a dataset consisting of the counts of transcriptional reads that map to all individual genes for each sample was made available in a public database (Sun et al., Sci Signal. 2015 Dec. 1;8 (405)).

We mined the literature using bioinformatics to create comparisons of CD6 expression in control vs. moderate vs. severe asthma patients, and our analysis demonstrated significant differences in levels of CD6 expression, with severe asthma patients expressing significantly higher levels of CD6 (FIG. 3B). Similar, though less pronounced increases in CD4 expression were also observed, with severe asthma patients expressing significantly higher levels of CD4 than moderate and healthy patients (FIG. 3A). Concomitant increase suggests increased presence of CD6⁺ T cells in the lungs of severe asthma patients Thus, these data suggest that severe asthma patients have increased CD6⁺ T cells in lungs due to increased T cell infiltration.

An analysis comparing expression of Th17 markers and cytokines in control vs. moderate vs. severe asthma patients demonstrated significant differences in levels of CCR6 (FIG. 3C), CCR4 (FIG. 3D), KLRB1 (FIG. 3E), IL-17A (FIG. 3F), and IL-17F (FIG. 3G) expression, indicating the increased presence of Th17 cells in severe asthma patients as well as an association between CD6 and Th17 cells.

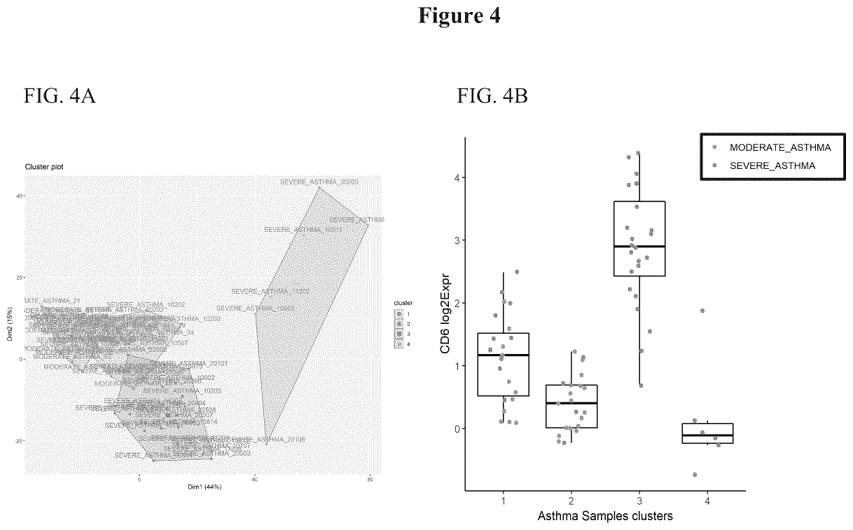

Utilizing the same dataset, an unbiased cluster analysis performed on all asthma patients, regardless of asthma type, demonstrated that a subgroup of severe asthmatic patients have both high CD6 and a similar gene expression profile, suggesting a distinct patient profile may be associated with high CD6 expression (FIG. 4A and FIG. 4B).

The authors are not aware of any prior analysis examining CD6 expression in this data set or of any prior reports that CD6 expression is increased in severe asthma. These data demonstrate that in severe asthma patients, which are poorly treated by steroids, CD6 expression is high in the lung. Thus, these data present for the first time evidence in support of the use of CD6 inhibition to treat severe asthma (e.g., caused by Th1/Th 17 T cells).

Notably, allergic asthma also includes a T cell component, albeit a Th2-mediated response that responds well to steroid treatment. Thus, we sought to determine whether CD6 might be a viable target for a treatment effective against both severe and allergic asthma, as this would be expected to: (1) minimize issues with patient compliance due to the adverse side-effects of long-term steroid use; (2) provide a convenient single agent therapy for all forms of asthma and; (3) provide a therapy that a patient can continue to use even when their asthma endotype shifts (e.g. Th2 to Th2/Th17 or Th1/Th17).

### Example 3

### CD6 Inhibition is a Viable Treatment For Allergic and Severe Asthma

Summary of Results: In a murine model of allergic asthma, CD6 blockade was demonstrated to be an effective immune modulator of Th2-mediated disease.

FIGS. 5A and 5B show the experimental groups and protocol, respectively, utilized in this allergic asthma experiment. Briefly, allergic asthma was induced via sensitization to ovalbumin (OVA) via vaccination with OVA/alum at Days 0 and 14 which induces an anti-OVA Th2-driven immune response. At day 23, 25, and 27, mice were treated with anti-mouse CD6 Sc-domain 1 antibody (mCD6D1), the mouse surrogate for EQ001, which binds to domain 1 of CD6 with similar characteristics to itolizumab, the anti-human CD6 antibody. Subsequently, sensitized mice were intranasally challenged with OVA on days 25-27, followed by termination at day 28 to evaluate cells and cytokines in the lung.

CD6 blockade during challenge resulted in decreased levels of Th2 cytokines, IL-4, IL-5 and IL-13 in bronchiolar lavage fluid (BALF) (FIG. 5C) accompanied by a modest reduction in lung cells, suggesting an inhibitory effect of CD6 blockade on Th2 responses.

To further support the ability of CD6 blockade to affect Th2 responses, we tested its affect in a classic Th2 model of OVA vaccination. FIGS. 6A and 6B show the experimental groups and protocol, respectively, utilized in this experiment. Briefly, mice were vaccinated with OVA/alum at days 0 and 14 and treated twice weekly with anti-CD6 starting at day-1 to day 16. At day 19, mice were sacrificed to examine the anti-OVA antibody response. The prophylactic CD6 blockade inhibited OVA-specific IgE production, demonstrating the effect of the CD6 pathway on Th2 responses (FIG. 6C).

Thus, taken together, these data demonstrate that CD6 blockade is a viable treatment, both acutely and prophylactically, for Th2-mediated allergic asthma. And, taken together the data presented herein coupled with the known ability of anti-CD6 antibodies such as EQ001 to inhibit activation and migration of Th1/Th17 type T cells, supports that CD6 inhibition (e.g., with EQ001) will be able to treat both severe and allergic asthma.

### Example 4

### Efficacy of Anti-CD6 Antibody in a Model of Severe Asthma

Mice are sensitized with 25 μg of HDM, intranasally on days 1, 3, and 5. Mice are then rested for 5 days and then subjected to 3 challenge sets involving 3 consecutive challenges with of 25 μg HDM with a rest of 4 days in between challenge sets. 25 μg HDM only in the next 2 challenges. A positive treatment control of dexamethasone (Dex) at a concentration of 4 mg/kg, or an anti-CD6 antibody test article is given intraperitoneally starting on the first day of the challenge and then is repeated every third day for Dex and every other day for the anti-CD6 antibody.

### Example 5

### Efficacy of Anti-CD6 Antibody in Asthma Models

Due to the ability of CD6 blockade to target multiple T cell subtypes, it is expected that CD6 inhibition will also show efficacy in a number of asthma models including HDM-induced, HDM+LPS induced, cockroach-induced, and Alternaria alternata-induced asthma models as well as asthma models generated in STAT6-/-mice which enable a Th1/Th17 phenotype. Accordingly, several models are utilized to test doses of anti-CD6 antibody in ranges of 600 μg down to 10 μg (Table 3).

TABLE 3
Additional Mouse Models

| Mouse model | Major Th | | |
|---|---|---|---|
| (citations) | response | Notes | |
| HDM-induced asthma | Th2/Th17 | Disease may be induced in two ways: | |
| (Woo et al., Sci Rep. | | 1) | Mice are sensitized by vaccination with |
| 2018 May 2; 8(1): 6925) | | | HDM + alum at days 0 and 14 to induce |
| | | | an anti-HDM response. During days 21- |
| | | | 23, mice are challenged intranasally daily |
| | | | with HDM eliciting recruitment of |
| | | | eosinophils and secretion of Th2 |
| | | | cytokines IL-4, IL-5, and IL-13 |
| | | 2) | Mice are sensitized intranasally or |
| | | | intratracheally with HDM in saline for 1 |
| | | | week. Subsequently, the mice are |
| | | | challenged intranasally 3-5 times weekly |
| | | | with HDM antigen for another 3-7 weeks |
| | | | prior to sacrifice. Intranasal challenge |
| | | | induces neutrophils, eosinophils and |
| | | | secretion of Th2 cytokines IL-4, IL-5, |
| | | | and IL-13 |

| | | |
|---|---|---|
| HDM + LPS-induced | Th1/Th17 | Mice are sensitized intranasally with HDM + LPS |
| asthma | | for one week followed by continued intranasal |
| (Daan De Boer et al., | | challenge 3x weekly for another 1-4 weeks to |
| Am J Respir Cell Mol Biol. | | induce lung inflammation characterized by |
| 2013 Mar.; 48(3): 382-9) | | |
| Alternaria alternata- | Th2 | Mice are sensitized by vaccination with spores of |
| induced asthma | | A. alternata/alum to induce an anti-spore |
| (Havaux et al., | | response. After several weeks, mice are serially |
| Clin Exp Immunol. | | challenged intranasally with spores eliciting |
| 2005 Feb.; 139(2): | | recruitment of eosinophils and secretion of Th2 |
| 179-88) | | cytokines IL-4, IL-5, and IL-13 |
| Cockroach-induced | Th2 | Mice are sensitized by vaccination with |
| asthma | | cockroach allergen + IFA to induce an anti- |
| | | cockroach response. After several weeks, mice |
| | | are serially challenged intranasally with |
| | | cockroach allergen eliciting recruitment of |
| | | eosinophils, secretion of Th2 cytokines IL-4, |
| | | IL-5, and IL-13, and chemokines |
| Asthma models in | Th1/Th17 | STAT6 signaling is required for Th2-type |
| STAT6−/− mice | | responses in asthma. Hence, deletion of this |
| (Valladoa et al., J Immunol. | | pathway abolishes the normally eosinophilic/Th2 |
| 2016 Dec. 15; 197(12): | | dominant response and biases the model to |
| 4541-4551) | | neutrophilic/Th1/Th17 type disease |

Results of these experiments will demonstrate that CD6 inhibition is effective at inhibiting Th1/Th17; Th2, and T2/Th17 forms of asthma, alike. Thus, the present disclosure supports the development of an anti-CD6 antibody for the treatment of severe and allergic asthma.

### Example 6

### Analysis of Tissue Samples

Tissue samples are collected following completion of the protocols in Example 4 and 5 and are analyzed according to the following procedures.

The bodyweights and clinical scores of all mice are collected every day as per approved Institutional Animal Care and Use Committee (IACUC) protocol.

Twenty-four hours after the final allergen challenge, mice are anesthetized and Bronchoalveolar lavage (BAL) is performed. The left bronchus in each mouse is tied off, and the right lobe is lavaged with 0.7 ml of sterile PBS to obtain BAL fluid. Total BAL cell numbers are determined using trypan blue staining and standard light microscopy, and 750,00 BAL fluid cells are cytospun onto clean glass slides for differential cell counts using Giemsa staining.

The BAL fluid is centrifuged to separate cells from supernatant. The supernatant is analyzed using standard cytokine assays to identify specific cell markers and/or cytokines while cells are analyzed by standard flow cytometry methods. Lavaged lungs are placed in fixative solution for 48 hours and then transferred to 70% ethanol until paraffin embedding for periodic acid-Schiff (PAS) staining, hematoxylin and eosin staining, and/or immunohistochemical or immunofluorescent staining for T cell markers (such as CD3, CD4, CD8), CD6, ALCAM and other known or potential ligands of CD6.

The various embodiments described above can be combined to provide further embodiments. All of the U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified, if necessary to employ concepts of the various patents, application and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. (canceled)

2. A method of inhibiting T cell-mediated pulmonary inflammation in a subject that has severe asthma comprising, administering to a subject an anti-CD6 antibody, or an antigen binding fragment thereof.

3-5. (canceled)

6. The method of claim 2, wherein: the asthma is refractory to steroid treatment, the asthma is a neutrophilic asthma, the asthma is a mixed inflammation asthma, the asthma is paucigranulocytic, or wherein the asthma is non-allergic asthma characterized by low or no blood eosinophils.

7. The method of claim 6, wherein the asthma is characterized by low or no blood eosinophils, and wherein the subject has blood eosinophils counts ≤300 cells/μl.

8-13. (canceled)

14. The method of claim 2, wherein the anti-CD6 antibody, or the antigen binding fragment thereof, is Itolizumab (EQ001), or an antigen binding fragment of EQ001.

15-17. (canceled)

18. The method of claim 2, wherein the anti-CD6 antibody, or the antigen binding fragment thereof, is a humanized antibody.

19. The method of claim 2, wherein the anti-CD6 antibody, or the antigen binding fragment thereof, is selected from an anti-CD6 antibody described on Table 2.

20. The method of claim 2, wherein the anti-CD6 monoclonal antibody is an antibody produced by secreting hybridoma IOR-TIA deposited with the ECACC as deposit No. ECACC 96112640; an antibody having the same sequence as said antibody produced by said secreting hybridoma; or an antibody having the same CDR sequences of said antibody produced by said secreting hybridoma.

21. The method of claim 2, wherein the anti-CD6 monoclonal antibody is Itolizumab (EQ001).

22. The method of claim 24, wherein the antigen binding fragment is selected from an Fv, a Fab, a (Fab′)2, a single chain Fv (scFv), a diabody, a triabody, a tetrabody, a bifunctional hybrid antibody, and a bispecific antibody.

23. The method of claim 2, wherein the anti-CD6 antibody, or the antigen binding fragment thereof, comprises one or more CDR sequence selected from SEQ ID NOS: 5-10.

24. The method of claim 2, wherein the anti-CD6 antibody, or the antigen binding fragment thereof, comprises heavy and light chain variable regions comprising amino acid sequences as set forth in SEQ ID NOs: 1 and 2.

25. The method of claim 24, wherein SEQ ID NOs: 1 and 2 are encoded by SEQ ID NOs: 3 and 4 respectively.

26-27. (canceled)

28. The method of claim 2, wherein the anti-CD6 antibody, or the antigen binding fragment thereof, comprises a VH sequence that is at least 80% identical to the amino acid sequence as set forth in SEQ ID NO: 1 and a VK sequence that is at least 80% identical to the amino acid sequence as set forth in SEQ ID NO: 2.

29. The method of claim 2, wherein the method further comprises administering one or more additional agents capable of treating, preventing, or attenuating one or more asthma related symptom.

30. (canceled)

31. The method of claim 29, wherein the additional agent is an immunosuppressant, a long-acting beta agonist. a short-acting beta agonist, or a combination thereof.

32. (canceled)

33. The method of claim 29, wherein the additional agent comprises albuterol.

34. (canceled)

35. The method of claim 29, wherein the additional agent comprises a corticosteroid.

36-37. (canceled)

38. The method of claim 29, wherein the additional agent comprises Ipratropium.

39-92. (canceled)


SEQUENCE LISTING (10 sequences)

SEQ ID NO: 1 - protein, 119 aa, synthetic construct
  REGION 1..119: note = humanized T1h VH sequence
EVQLVESGGGLVKPGGSLKLSCAASGFKFSRYAMSWVRQAPGKRLEWVATISSGGSYIYYPDSVKGRFTISRDNVKNTLYLQMSSLRSEDTAMYYCARRDYDLDYFDSWGQGTLVTVSS

SEQ ID NO: 2 - protein, 107 aa, synthetic construct
  REGION 1..107: note = humanized T1h VL sequence
DIQMTQSPSSLSASVGDRVTITCKASRDIRSYLTWYQQKPGKAPKTLIYYATSLADGVPSRFSGSGSGQDYSLTISSLESDDTATYYCLQHGESPFTLGSGTKLEIK

SEQ ID NO: 3 - DNA, 357 bp, synthetic construct
  misc_feature 1..357: note = humanized T1h VH nucleic acid sequence
  source 1..357: mol_type = other DNA
gaagtgcagctggtggagtctgggggaggcttagtgaagcctggagggtccctgaaactctcctgtgcagcctctggattcaagtttagtagatatgccatgtcttgggttcgccaggctccggggaagaggctggagtgggtcgcaaccattagtagtggtggtagttacatctactatccagacagtgtgaagggtcgattcaccatctccagagacaatgtcaagaacaccctgtatctgcaaatgagcagtctgaggtctgaggacacggccatgtattactgtgcaagacgagattacgacctggactactttgactcctggggccaaggcacccttgtcaccgtctcctca

SEQ ID NO: 4 - DNA, 321 bp, synthetic construct
  misc_feature 1..321: note = humanized T1h VL nucleic acid sequence
  source 1..321: mol_type = other DNA
gacatccagatgacccagtctccatcctccctgtctgcatcggtgggagacagagtcactatcacttgcaaggcgagtcgggacattagaagctatttaacctggtaccagcagaaaccagggaaagctcctaagaccctgatctattatgcaacaagcttggcagatggggtcccgtcgagattcagtggcagtggatctgggcaagattattctctcaccatcagcagcctggagtctgacgatacagcaacttactactgtctacaacatggtgagagtccattcacgctcggctcggggaccaagctggaaatcaaa

SEQ ID NO: 5 - protein, 10 aa, synthetic construct
  REGION 1..10: note = humanized T1h VH CDR1
GFKFSRYAMS

SEQ ID NO: 6 - protein, 17 aa, synthetic construct
  REGION 1..17: note = humanized T1h VH CDR2
TISSGGSYIYYPDSVKG

SEQ ID NO: 7 - protein, 10 aa, synthetic construct
  REGION 1..10: note = humanized T1h VH CDR3
RDYDLDYFDS

SEQ ID NO: 8 - protein, 11 aa, synthetic construct
  REGION 1..11: note = humanized T1h VL CDR1
KASRDIRSYLT

SEQ ID NO: 9 - protein, 7 aa, synthetic construct
  REGION 1..7: note = humanized T1h VL CDR2
YATSLAD

SEQ ID NO: 10 - protein, 7 aa, synthetic construct
  REGION 1..7: note = humanized T1h VL CDR3
LQHGESP